SEQUENCE LISTING (23 sequences)

SEQ ID NO: 1 - protein, 9 aa, Artificial
  chemically synthesized
  MISC_FEATURE (1)..(9): arginine-rich peptide effective in internalizing a bile acid moiety
RANARANAR

SEQ ID NO: 2 - protein, 13 aa, Artificial
  chemically synthesized
  MISC_FEATURE (1)..(13): arginine-rich peptide effective in internalizing a bile acid moiety
RANARANARANAR

SEQ ID NO: 3 - protein, 17 aa, Artificial
  chemically synthesized
  MISC_FEATURE (1)..(17): arginine-rich peptide effective in internalizing a bile acid moiety
RANARANARANARANAR

SEQ ID NO: 4 - protein, 21 aa, Artificial
  chemically synthesized
  MISC_FEATURE (1)..(21): arginine-rich peptide effective in internalizing a bile acid moiety
RANARANARANARANARANAR

SEQ ID NO: 5 - protein, 13 aa, Artificial
  chemically synthesized <s300> Jones, Determinants of human immunodeficiency virus type 1 escape from the primary CD8+ cytotoxic T lymphocyte response J. Exp. Med. 200 10 1243-1256 2004 (48)..(60)
GRKKRRQRRRPPQ

SEQ ID NO: 6 - protein, 13 aa, Artificial
  chemically synthesized
  MISC_FEATURE (1)..(13): tat protein form human immunodeficiency virus 1 <s300> Lorenzo, Genetic characterization of human immunodeficiency virus type 1 Tat before and after highly active antiretroviral therapy AIDS Res. Hum. Retroviruses 20 10 1108-1112 2004 (48)..(60)
GRRRRRRRRRPPQ

SEQ ID NO: 7 - protein, 17 aa, Artificial
  chemically synthesized
  MISC_FEATURE (1)..(17): rev protein of human immunodeficiency virus 1 <s300> Liu, Selection on the human immunodeficiency virus type 1 proteome following primary infection J. Virol. 80 19 9519-9529 2006 (34)..(50)
TRQARRNRRRRWRERQR

SEQ ID NO: 8 - protein, 15 aa, Artificial
  chemically synthesized
  MISC_FEATURE (1)..(15): hypothetical protein FHVgp1 (Flock house virus) <s300> Dasgupta, Nucleotide sequences of three Nodavirus RNA2′s the messengers for their coat protein precursors Nucleic Acids Res. 17 18 7525-7526 1989 (35)..(49)
RRRRNRTRRNRRRVR

SEQ ID NO: 9 - protein, 19 aa, Artificial
  chemically synthesized
  MISC_FEATURE (1)..(19): hypothetical protein BMVs3gp2 (Brome mosaic virus); Viruses; ssRNA positive-strand viruses, no DNA stage; Bromoviridae; Bromovirus <s300> Ahlquist, Complete nucleotide sequence of brome mosaic virus RNA3 J. Mol. Biol. 153 1 23-28 1981 (7)..(25)
KMTRAQRRAAARRNRWTAR

SEQ ID NO: 10 - protein, 13 aa, Artificial
  chemically synthesized
  protein Rex (1)..(13): mRNA transport; Nuclear protein; Phosphorylation RNA-binding; Transport <s300> Shimotohno, Complete nucleotide sequence of an infectious clone of human T-cell leukemia virus type II an open reading frame for the protease gene Proc. Natl. Acad. Sci. U.S.A. 82 10 3101-3105 1985 (4)..(16)
TRRQRTRRARRNR

SEQ ID NO: 11 - protein, 26 aa, Artificial
  chemically synthesized
  MISC_FEATURE (1)..(26): Binding-protein-dependent transport systems inner membrane component; Clostridium phytofermentans ISDg
LIKKALAALAKLNIKLLYGASNLTWG

SEQ ID NO: 12 - protein, 27 aa, Artificial
  chemically synthesized
  MISC_FEATURE (1)..(26): Chain A Transportan; Chain A Structure Of Transportan In Phospholipid Bicellar Solution
GWTLNSAGYLLGKINLKALAALAKKIL

SEQ ID NO: 13 - protein, 24 aa, Artificial
  chemically synthesized
  Chain A Transportan (1)..(24): Chain A, Structure Of Transportan In Phospholipid Bicellar Solution <s300> Barany-Wallje, NMR solution structure and position of transportan in neutral phospholipid bicelles FEBS Lett. 567 2 265-269 2004 (2)..(25)
LNSAGYLLGKINLKALAALAKKIL

SEQ ID NO: 14 - protein, 25 aa, Artificial
  chemically synthesized
  Chain A Transportan (1)..(25): Chain A, Structure Of Transportan In Phospholipid Bicellar Solution <s300> Barany-Wallje, NMR solution structure and position of transportan in neutral phospholipid bicelles FEBS Lett. 567 2 265-269 2004 (2)..(25)
GWTLNSAGYLLGKLKALAALAKKIL

SEQ ID NO: 15 - protein, 21 aa, Artificial
  chemically synthesized
  Chain A Transportan (1)..(21): Chain A, Structure Of Transportan In Phospholipid Bicellar Solution <s300> Barany-Wallje, NMR solution structure and position of transportan in neutral phospholipid bicelles FEBS Lett. 567 2 265-269 2004 (5)..(25)
AGYLLGKINLKALAALAKKIL

SEQ ID NO: 16 - protein, 22 aa, Artificial
  chemically synthesized
  Chain A Transportan (1)..(21): Chain A, Structure of Transportan In Phospholipid Bicellar Solution <s300> Barany-Wallje, NMR solution structure and position of transportan in neutral phospholipid bicelles FEBS Lett. 567 2 265-269 2004 (5)..(25)
LNSAGYLLGKLKALAALAKKIL

SEQ ID NO: 17 - protein, 19 aa, Artificial
  chemically synthesized
  Chain A Transportan (1)..(19): Chain A, Structure Of Transportan In Phospholipid Bicellar Solution <s300> Barany-Wallje, NMR solution structure and position of transportan in neutral phospholipid bicelles FEBS Lett. 567 2 265-269 2004 (7)..(24)
AGYLLGKLKALAALAKKIL

SEQ ID NO: 18 - protein, 16 aa, Artificial
  chemically synthesized
  MISC_FEATURE (1)..(16): hox 6 (Saccoglossus kowalevskii) <s300> Aronowicz, Hox gene expression in the hemichordate Saccoglossus kowalevskii and the evolution of deuterostome nervous systems Integr. Comp. Biol. 46 890-901 2006 (190)..(205)
RQIKIWFQNRRMKWKK

SEQ ID NO: 19 - protein, 9 aa, Artificial
  chemically synthesized
  MISC_FEATURE (1)..(9): Aspartic acid rich peptide; Anionic moiety effective in internalizing a bile acid or derivative
EANAEANAE

SEQ ID NO: 20 - protein, 13 aa, Artificial
  chemically synthesized
  MISC_FEATURE (1)..(13): Aspartic acid rich peptide; Anionic moiety effective in internalizing a bile acid or derivative
EANAEANAEANAE

SEQ ID NO: 21 - protein, 17 aa, Artificial
  chemically synthesized
  MISC_FEATURE (1)..(17): Aspartic acid rich peptide; Anionic moiety effective in internalizing a bile acid or derivative
EANAEANAEANAEANAE

SEQ ID NO: 22 - protein, 21 aa, Artificial
  chemically synthesized
  MISC_FEATURE (1)..(21): Aspartic acid rich peptide; Anionic moiety effective in internalizing a bile acid or derivative
EANAEANAEANAEANAEANAE

SEQ ID NO: 23 - protein, 12 aa, Artificial
  chemically synthesized
  MISC_FEATURE (1)..(12): (Corynebacterium efficiens YS-314); putative iron transport membrane protein <s300> Nishio, Comparative complete genome sequence analysis of the amino acid replacements responsible for the thermostability of Corynebacterium efficiens Genome Res. 13 7 1572-1579 2003 (211)..(221)
AAVLLPVLLAAP

(19) United States
(12) Patent Application Publication
(10) Pub. No.: US 2008/0026077 A1
(43) Pub. Date: Jan. 31, 2008

(54) METHODS AND COMPOSITIONS OF GENE DELIVERY AGENTS FOR SYSTEMIC AND LOCAL THERAPY

(76) Inventors: John Hilfinger, Ann Arbor, MI (US); Phillip Kish, Ann Arbor, MI (US); Blake Roessler, Ann Arbor, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/608,370

(22) Filed: Dec. 8, 2006

Related U.S. Application Data
(63) Continuation-in-part of application No. 10/706,738, filed on Nov. 12, 2003.
(60) Provisional application No. 60/425,379, filed on Nov. 12, 2002.
(60) Provisional application No. 60/748,390, filed on Dec. 8, 2005.

(51) Int. Cl. C07K 14/00; A61K 31/19; A61K 31/195; A61K 31/351; A61K 31/397; A61K 31/40; A61K 31/401; A61K 31/403; A61K 31/405; A61K 31/407; A61K 31/415; A61K 31/4164; A61K 31/43; A61K 31/4355; A61K 31/44; A61K 31/47; A61K 31/485; A61K 31/4965; A61K 31/497; A61K 31/519; A61K 31/535; A61K 31/545; A61K 31/55; A61K 31/60; A61K 31/704; A61K 33/06; A61K 33/08; A61K 33/26; A61K 38/00; A61P 43/00

(52) U.S. Cl. 424/648; 424/687; 424/690; 424/692; 514/159; 514/165; 514/2; 514/210.17; 514/212.07; 514/224.2; 514/230.2; 514/249; 514/252.17; 514/253.04; 514/253.08; 514/254.04; 514/255.06; 514/26; 514/262.1; 514/282; 514/283; 514/300; 514/314; 514/361; 514/368; 514/383; 514/389; 514/394; 514/409; 514/411; 514/412; 514/413; 514/415; 514/423; 514/459; 514/561; 514/568; 514/789; 530/324

(57) ABSTRACT

A method is provided for the delivery of a therapeutic to epithelial cells through the use of a bile acid conjugated to a peptide, the peptide being ionically charged at physiological pH. The complex is well suited for oral and other forms of therapeutic administration of therapeutic drugs known in the art in order to exact systemic and/or localized effect. Intestinal epithelial cells, as well as non-epithelial cells within the gastrointestinal tract and other target cells receive with greater efficiency a charged therapeutic when delivered with an oppositely charged bile acid conjugate (BAC) through oral administration, direct injection, or infusive administrations, thereby increasing bioavailability.

### CROSS-REFERENCE TO RELATED APPLICATIONS

This application is a continuation-in-part application of U.S. patent application Ser. No. 10/706,738 filed Nov. 12, 2003, which claims priority of U.S. Provisional Patent Application Ser. No. 60/425,379 filed Nov. 12, 2002, the contents of these priority documents are hereby incorporated by reference; this application also claims priority benefit of U.S. Provisional Patent Application Ser. No. 60/748,390 filed Dec. 8, 2005, the contents of which are hereby incorporated by reference.

### FIELD OF THE INVENTION

The invention relates in general to compositions and methods that enhance the delivery of low bioavailability therapeutics or genes across epithelial membranes, including, for example, skin, the gastrointestinal epithelium and the bronchial epithelium, and in particular to the use of a bile acid derivative conjugate to enhance transport of the therapeutic across the target cell membrane.

### BACKGROUND OF THE INVENTION

Methods for increasing drug absorption and bioavailability have garnered considerable attention for enhancing therapeutic levels of small molecule drugs (anionic, cationic or neutrally charged) or nucleic acids, peptides and proteins to treat various mammalian diseases. In addition to the delivery of drugs, gene mediated therapy holds the promise of treating or correcting underlying genetic deficiencies or defects without long term continuous treatment. However, the delivery of genetic material into a multi-celled organism has proven more difficult than initially imagined. A variety of techniques have been developed to accomplish in vivo transformation of cells including direct injection of nucleic acid or a particle decorated with nucleic acid directly into cells, recombinant viruses, liposomes and receptor mediated endocytosis.

In attempting to develop lower cost modes of administration that are likely to enhance patient compliance, intestinal absorption has been recognized as an attractive site for in vivo gene therapy owing to the ease of access through oral or rectal routes. Oral therapy is easy to administer, generally the least expensive, and has good patient compliance for dosing. Difficulties in this method include therapeutic insolubility, and penetrating the mucus layer, which may further reduce the amount of the cellular exposure and reduce absorption efficiency. Further, the intestine routinely degrades large quantities of foreign nucleic acid ingested and drug species as part of foodstuffs. DNAses and RNAses in the intestinal tract represent a significant barrier to the entry of intact and functional nucleic acids to intestinal tract cells. As a result, many useful drugs are limited in administration routes to intravenous or intramuscular injection.

The intestine routinely degrades large amounts of foreign DNA and proteins ingested in our food. The presence of proteases and other digestive enzymes in the intestinal tract can provide a significant barrier to entry of protein and peptide therapeutics into intestinal tract cells.

Gene therapy has garnered considerable attention as a method to treat various human diseases by the enhancement of protein production. These include gene replacement or gene augmentation.

U.S. Pat. No. 6,225,290 represents an effort to deliver bare nucleic acid sequences through laparotomy, oral or suppository administration but is silent as to nucleic acid protection and overcoming the above-stated problems of intestinal administration. It is generally agreed that an oral administration of nucleic acids represents the least expensive and most likely route for the attainment of patient compliance with dosing requirements.

While oral administration is generally recognized as the superior route, little attention has been paid to methodologies and packaging of DNA to preclude intestinal degradation. U.S. Pat. No. 6,500,807 is representative of an attempt to produce a protective coating of carbohydrate around a nucleic acid to facilitate oral administration. It would be advantageous to deliver nucleic acids in a form other than micelles to facilitate conventional pharmaceutical compounding.

Thus, there exists a need for improved gene and small molecule delivery agents amenable to efficient transfection of target cells and capable of inducing systemic and/or local transfection and therapeutic delivery.

### SUMMARY OF THE INVENTION

A compound having the formula:
RC(O)—X-Z (I)

where RC(O)— is a reaction product of bile acid (5β-CHOLANIC ACID-3α, 7α, -DIOL) or a derivative of the form RCOOH and the derivative is lithocholic acid, deoxycholic acid, chenodeoxycholic acid, ursodeoxycholic acid, ursocholic acid, hyocholic acid, hyodeoxycholic acid, murocholic acid, dehydrocholic acid, 7-ketodeoxycholic acid, dehydrocholic acid, diketocholanic acid, triketocholanic acid, isolithocholic acid, ketolithocholic acid, dehydrolithocholic acid, allocholanic acid, or a salt thereof;

where Z is a 1 to 50 amino acid residue chain having a net charge at physiological pH through at least 20 residue percent basic residues of arginine, lysine, or a combination thereof; at least 20 residue percent acid residues of aspartic acid, glutamic acid, or a combination thereof; or a privileged lysine containing internalization moiety of any one of SEQ ID Nos. 11-18;

where X is a nullity or has a structure prior to reaction with RCOOH of M₁-B-M₂, where M₁ is an amine, CH₂═CH—, iodo-, bromo-, chloro-, or N₂—; where M₂ is amine, CH₂═CH—, iodo-, bromo-, chloro-, or N₂—, carboxylate, thionyl chloride, or acid chloride; and where B is a carbon backbone of 1 to 5 amino acid residues, C₂-C₅ alkyl, C₂-C₁₆ alkenyl, C₂-C₁₆ aryl, and C₂-C₁₆ heteroaromatic compounds, where the heteroatom is O, N, or S; and

wherein the compound (I) has a net anionic or cationic charge.

The compound (I) has a net anionic or cationic charge. By associating the compound (I) with a therapeutic of an opposite ionic charge at physiological pH, the bioavailability of the therapeutic is increased. By controlling the concentration of the compound (D, a micelle is formed by the compound (I) internalizing the therapeutic for delivery. A process of administration of a composition containing a compound (I) and a therapeutic is also provided.

### BRIEF DESCRIPTION OF THE DRAWINGS

FIG. 1 is transmission electron microscopy (TEM) of a bile acid conjugate (BAC)/plasmid DNA (pDNA) complex;

FIGS. 2a-2d are relative percent particle size bar graphs of: (FIG. 2a) pDNA; (FIG. 2b) pDNA and 50 μg/ml BAC initially upon mixing and corresponding to a negatively charged complex; (FIG. 2c) pDNA and 200 μg/ml BAC initially upon mixing and corresponding to a neutrally charged complex; and (FIG. 2d) the complex depicted in FIG. 2c after two hours of mixing;

FIGS. 3a-3c are relative percent particle size bar graphs of inventive BAC-pDNA particles having a relative ratio of BAC:pDNA of 80, 699:1 in: (FIG. 3a) water; (FIG. 3b) simulated intestinal fluid; and (FIG. 3c) simulated gastric fluid;

FIG. 4 is a plot of the zeta potential measured as a function of conjugating agent:DNA mol ratio where (●) indicates addition of further conjugating agent after particle formation and (▴) indicates the addition of additional nucleic acid after particle formation;

FIG. 5 is an electrophoretic gel showing the relative mobility of nucleic acid, alone and in combination with at least one of conjugating agent, polyaspartic acid and DNAse I;

FIG. 6 is a bar graph showing in vitro transfection of Hela cells with BAC-luciferase pDNA at various conjugating agent:pDNA ratios;

FIG. 7 is a plot of inventive complex single pass absorption for neutrally charged and positively charged radiolabeled BAC-pDNA as a function of time where filled symbols indicate jejunal absorption and open symbols indicate ileal absorption;

FIG. 8 represents octanol partition of enelaprilat with inventive compound B11;

FIG. 9 represents in vitro plasma concentration of intestinally absorbed enelaprilat (3 mg dose) with and without an added inventive compound;

FIG. 10 represents in vitro plasma concentration of intestinally absorbed enelaprilat (0.3 mg dose) with and without an added inventive compound;

FIG. 11 represents the in vivo plasma concentration of an IV dose of zanamivir (0.1 mg dose);

FIG. 12 represents in vivo plasma concentration of intestinally absorbed zanamivir (0.05 mg dose) with and without inventive compound BAC A6 (N-A6 motif of chenoxycholamide);

FIG. 13 represents in vivo plasma concentration after oral dosing of zanamivir (4 mg/kg) with and without BAC_A6 (N-A6 motif of chenoxycholamide) (12 mg/kg) in fasted mice;

FIG. 14 represents in vitro plasma concentration after oral dosing of zanamivir (40 mg/kg) with and without BAC-A6 (N-A6 motif of chenoxycholamide) (120 mg/kg) in fasted mice;

FIG. 15 represents in vivo plasma concentration after oral dosing of zanamivir (10 mg/kg) with and without BAC-A6 (N-A6 motif of chenoxycholamide) (30 mg/kg) in fed mice;

FIG. 16 represents in vivo plasma concentration after oral dosing of zanamivir (10 mg/kg) at increasing levels of BAC-A6 (N-A6 motif of chenoxycholamide) in fed mice;

FIG. 17 represents sample pharmacokinetics of alendronate dosed in fasted animals at 4 mg/kg and increased plasma concentrations when dosed in the presence of BAC A6 (N-A6 motif of chenoxyholamide);

FIG. 18 represents sample pharmacokinetics of alendronate dosed in fed animals at 4 mg/kg and increased plasma concentrations when dosed in the presence of BAC A6 (N-A6 motif of chenoxycholamide);

FIG. 19 represents sample pharmacokinetics of alendronate dosed in fed animals at 0.4 mg/kg and increased plasma concentrations when dosed in the presence of SAC A6 (N-A6 motif of chenoxycholamide);

FIG. 20 illustrates absorption of alendronate in fasted mice in vivo plasma concentration is increased by BAC A6 (N-A6 motif of chenoxycholamide) in a dose responsive manner;

FIG. 21 represents the area under the curve (AUC) increases corresponding in vivo plasma concentrations to increasing molar ratios of BAC A6 (N-A6 motif of chenoxycholamide); and

FIG. 22 represents the effect of increasing molar ratios of multiple BACs on methotrexate plasma concentrations in mice following oral dosing;

### DETAILED DESCRIPTION OF THE PREFERRED EMBODIMENTS

The present invention has utility in facilitating therapeutic or nucleic acid delivery into target cells and in particular epithelial cells. The invention involves methods and products for oral, parenteral, or topical therapeutic delivery, the delivery of a therapeutic or nucleic acid according to the present being for either systemic or localized therapy. An inventive composition is a complex with the therapeutic agent, which increases the efficiency of absorption of the therapeutic into cells relative to the bare therapeutic agent. Depending on the nature of the therapeutic or nucleic acid sequence, these contain, in non-covalently bound form, one or more substances having an affinity for nucleic acid, which are capable of increasing the efficiency of absorption of the complexes into the cells. Cells of a mammalian subject, either intestinal epithelia after oral delivery, or cells in other organs after parenteral, inhalational or topical delivery, absorb the inventive composition which penetrates the cell and is distributes into the organ and/or bloodstream the therapeutic or nucleic acid to provide a therapeutic effect. An inventive composition is optionally delivered via the intestinal lumen in a variety of ways, including through timed-release capsules, thereby obtaining a simple, noninvasive method of drug delivery for therapy. These complexes can also be delivered to other organs of the body in a variety of ways, including direct injection, infusion or topical administration. After oral delivery, the intestinal epithelial cells provide short or long term therapies for diseases illustratively including metabolic disorders, endocrine disorders, circulatory disorders, coagulation disorders, cancer, bacterial infection, eukaryotic infection, viral infection, and gastrointestinal disease.

Bile acids conjugated with peptides retain the capability of forming micellar structures, and assist the delivery of a therapeutic agent across the mucus and unstirred water layer of the intestine as well as the cutaneous barrier of the skin. A conjugated bile acid or derivative thereof is expected to solve problems of degradation or poor bioavailability of a piggybacked therapeutic to be delivered via oral or rectal administration.

As used herein, a “gene” is defined to be an isolated nucleic acid molecule of greater than twenty nucleotides. A gene operative herein is recognized to be one that illustratively replaces or supplements a desired function, or achieves a desired effect such as the inhibition of tumor growth or induction of an immune response to the gene itself or a polypeptide transcribed therefrom. It is appreciated that a nucleic acid molecule according to the present invention illustratively includes plasmids, vectors, and external guide sequences for RNAase, ribozymes, DNA, RNA, and miRNA. Antisense nucleic acids sequences are also administered according to the present invention. A gene is generally under the control of an appropriate promoter, which may be inducible, repressible, or constitutive. Promoters can be general promoters, yielding expression in a variety of mammalian cells, or cell specific, or even nuclear versus cytoplasmic specific. Viral promoters such as CMV are also operative herein. These are known to those skilled in the art and can be constructed using standard molecular biology protocols.

As used herein, “therapeutic”, synonymously described as a therapeutic agent is defined to include an organic molecular or salt thereof having a an intestinal bioavailability that is less than that of intravenous bioavailability as detailed in Remington The Science and Practice of Pharmacy, 20^{th} ed. (2000) pages 1098-1126, and 1146, or an organic molecule or salt thereof in which the pharmacokinetic and or pharmacodynamic profile is altered when co administered with an inventive compound. A typical feature of an inventive therapeutic is an ionic charge at physiological pH and a monomeric molecular weight of less than 2,000 Daltons, as such dimeric through tetrameric conjugated therapeutic that exceeds the molecular weight of 2,000 Daltons is considered to be operative herein.

In a preferred embodiment administration is oral and targeted to transfect intestinal epithelial cells.

As used herein, a “subject” is defined as a mammal and illustratively includes humans, non-human primates, horses, goats, cows, sheep, pigs, dogs, cats, and rodents. The methods and compounds of the present invention are administered in therapeutically effective amounts.

As used herein, a “therapeutically effective amount” is defined to include an amount necessary to delay the onset of, inhibit the progress of, relieve the symptoms of, or reverse a condition being treated; induce an immune response to the delivered gene or a polypeptide encoded thereby or regulate the expression of an existing cellular product. The therapeutically effective amount is one that is less than that that produces medically unacceptable side effects. It is appreciated that a therapeutically effective amount varies with a number of factors illustratively including subject age, condition, sex and the nature of the condition being treated. It is further appreciated that determining a therapeutically effective dose is within the knowledge of one of ordinary skill in the art.

As used herein, however, the term “peptide” is intended to include mimetics and is used synonymously with polypeptide.

As used herein, however, the term “amino acid chain” is intended to include at least one amino acid, and peptide mimetics.

The term “amino acid” is intended to include L- and D-form amino acids, and non-naturally occurring amino acids.

The therapeutic and nucleic acid of the present invention are illustratively administered to a subject at dosage levels in the range of about 0.005-500 mg/kg/day of bile acid or derivative thereof conjugating agent combined with about 5×10⁻⁶-500 mg/kg/day of therapeutic or nucleic acid per day. The general ratio of the amount of conjugating agent to the therapeutic or nucleic acid ranges from about 1 a molar ratio of 0.5:1-500,000:1 in the composition administered to a subject.

The absorption enhancing portion of an inventive compound is preferably a bile acid conjugated with an ionic amino acid chain linked to a bile acid steroid backbone (BAC). The bile acid moiety acts to target the therapeutic agent to the mucosal surface in the lumen of the intestine and assist in the cellular internalization of the complex. Short amino acid chains rich in arginine or lysine (cationic), or aspartic acid or glutamic acid (anionic) coupled thereto provide an affinity for opposite ionic charged therapeutic, and assist in cellular internalization of the composition. A short amino acid chains is one that contains from 1 to 50 amino acid residues of which at least 20% are one of the aforementioned residues. Typically, 1 to 50 such residues are found within an amino acid chain. It is appreciated that an amino acid chain inclusive of both cationic and anionic amino acid residues offsets the charge attributes of one another and therefore increases peptide chain length with little advantage. As such, preferably an amino acid chain includes only cationic, cationic and neutral, anionic, or anionic and neutral charge amino acid residues.

Conjugation of the amino acid chain to the bile salt yields either a cationic or anionic compound and is made through the hydroxyl groups in the 3, 7, or 12 positions of the bile acid steroid nucleus, or on the 24-carboxyl group of the bile acids.

The invention includes a bile acid conjugate (BAC), synonymously referred to as a bile acid compound having the formula:
RC(O)—X-Z (I)

where RC(O)— is a reaction product of bile acid (5β-CHOLANIC ACID-3α, 7α, -DIOL) or a derivative of the form RCOOH and is lithocholic acid, deoxycholic acid, chenodeoxycholic acid, ursodeoxycholic acid, ursocholic acid, hyocholic acid, hyodeoxycholic acid, murocholic acid, dehydrocholic acid, 7-ketodeoxycholic acid, dehydrocholic acid, diketocholanic acid, triketocholanic acid, isolithocholic acid, ketolithocholic acid, dehydrolithocholic acid, allocholanic acid, salts of any of the preceding or a combination of any of the preceding. Preferably, the derivative substituent forms a linkage to Z directly through the carbonyl carbon atom of RCOOH or indirectly to Z via an intermediate X linker. More preferably, the BAC has a net anionic or cationic charge. It is appreciated that in addition to the above identities for RC(O)—, a given hydrogen of R is optionally replaced with a C₁-C₂₄ alkyl to modify the lipophilicity of the resultant BAC.

The linker X is a nullity or has a structure prior to reaction with RCOOH of M₁-B-M₂, where the first moiety M₁ is reactive with the bile acid carbonyl carbon of RCOOH and a second moiety M₂ is reactive with an ionic amino acid chain Z through the Z amine, carboxyl, or a side chain moiety. M₁ is an amine, CH₂═CH—, iodo-, bromo-, chloro-, or N₂—; where M₂ is amine, CH₂═CH—, iodo-, bromo-, chloro-, or N₂—, carboxylate, thionyl chloride, or acid chloride; and where B is a carbon backbone of 1 to 5 amino acid residues, C₂-C₁₆ alkyl, C₂-C₁₆ alkenyl, C₂-C₁₆ aryl, and C₂-C₁₆ heteroaromatic compounds, where the heteroatom is O, N, or S. Substituents extending from a linker backbone are provided to modify the lipophilicity of an inventive conjugate, or tether a dye or spectroscopic marker. With the inclusion of a linker X, care should be taken to limit both the molecular weight and the hydrophilicity of the linker in order to retain the ability to cross cellular membranes. Typically, the linker moiety is reactive with the bile acid carbonyl carbon to illustratively form an amide, ether, ester, sulfonyl, or other hydrolyzable bond. The Z amino acid reactive moiety of the linker is dependent upon the amino acid moiety to be bound thereto namely, an alpha amine or carboxyl carbon and includes an amine, a carboxyl, an acid chloride, and a sulfonyl chloride. Suitable chemistries for a variety of potential reaction moieties are found in Comprehensive Organic Transformations, R. C. Larock, John Wiley & Sons 1999 and include condensation reactions between an amine and carboxylate, reductive amination with a linker ketone in the presence of a nickel catalyst and hydrogen, acid chloride reaction with a peptide amine group, and sulfonyl chloride reaction with a peptide amine group.

A substituent is optionally provided pendent from the linker backbone. The substituent illustratively includes a radioactive atom, a magnetic spectroscopically active marker and an organic dye. A radioactive atom is alternatively operative as a marker in isotope studies such as positron emission tomography, single photon emission computer tomography, radiological studies and the like. Common radio-isotopes used in medical imaging illustratively include 1231, 99 mTc, and other chelated radioisotopes as detailed in U.S. Pat. No. 6,241,963. Spectroscopically active markers include NMR/MRI active contrast enhancing moieties known to the art such as gadolinium, as detailed in Contrast Agents 1: Magnetic Resonance Imaging (Topics in Current Chemistry, 221) by Werner Krause, Springer Verlag, Berlin, Germany. Organic dyes, while recognized to have potentially distinct NMR/MRI signatures, are provided to yield an optically active spectroscopic signature suitable for biopsy, surgical identification, or preclinical studies of tissue treated by an inventive compound.

A linker X is provided with the proviso that any charge associated with a linker that is accounted for in the overall charge state of the bile acid (I). A non-zero length linker amino acid chain is preferably provided in instances of steric hindrance, associated with the peptide Z or to utilize a synthetic chemistry scheme where a linker X is bound to R, and then amino acid chain Z added by a peptide coupling reaction with appropriate blocking groups added to preclude side reactions; Z is a net ionic amino acid chain up to and including 50 amino acid residues long.

Cationic amino acid chains operative herein as the Z moiety in formula (I) illustratively include a single arginine residue; a 2 to 50 residue oligopeptide that contains at least 20 residue percent arginine, or at least 20 residue percent lysine; or a privileged transport sequence such as transportan or penetratin sequences. Preferably, more than 30 residue percent arginine or 30 residue percent lysine is present. Most preferably Z is less than 25 total residues in length and more than 35 residue percent arginine or 45 residue percent lysine. Z amino acid residue chains of 3 to 15 residues with at least 50 residue percent of lysine and or arginine are particularly well suited for the delivery of anionic therapeutics complexed therewith in a charge neutralizing amount. Specific examples of cationic Z moieties effective in internalizing a bile acid moiety R and a coadministered therapeutic include wholly arginine or wholly lysine oligopeptides having a length of from 1 to 12 residues, synthetic residues (RANA)ₙR where n is an integer 2-5 (SEQ ID NOS. 1-4, respectively) and conventional arginine rich protein internalization proteins (M. Peitz et al. PNAS USA 2002; 99:4489-4494; D. Jo et al. Nature Biotech. 2001; 19:292-933) such as

(SEQ ID NO.5)

GRKKRRQRRRPPQ (TAT 48-60)

(SEQ ID NO.6)

GRRRRRRRPRPPQ (R9-TAT)

(SEQ ID NO.7)

TRQARRNRRRRWRERQR (HIV-1 Rev 34-50)

(SEQ ID NO.8)

RRRRNRTRRNRRRVR (FHV coat 35-49)

(SEQ ID NO.9)

KMTRAQRRAAARRNRWTAR (BMVgag7-25)

(SEQ ID NO.10)

TRRQRTRRARRNR (HTLV-II Rex 4-16);

privileged lysine containing protein internalization peptides (A. Muratovska et al., FEBS Let. 2004; 558:63-68) such as transportan,

(SEQ ID NO.11)

LIKKALAALAKLNIKLLYGASNLTWG;

and alternative amino acid composition for transportan and its deletion analogs which maintain membrane transduction properties (Soomets et al. Biochim Biophys Acta. 2000 Jul. 31; 1467(1):165-76)):

(SEQ ID NO.12)

GWTLNSAGYLLGKINLKALAALAKKIL (transportan)

(SEQ ID NO.13)

LNSAGYLLGKINLKALAALAKKIL (transportan7)

(SEQ ID NO.14)

GWTLNSAGYLLGKLKALAALAKKIL (transportan9)

(SEQ ID NO.15)

AGYLLGKINLKALAALAKKIL (transportan10)

(SEQ ID NO.16)

LNSAGYLLGKLKALAALAKKIL (transportan12);
and

(SEQ ID NO.17)

AGYLLGKLKALAALAKKIL (transportan14);
and penetratin

(SEQ ID NO.18)

RQIKIQFQNRRMKWKK (Atennapedia 43-58 - pentratin).

As abbreviated above TAT denotes HIV-1 transaction of transcription, FHV denotes flock house virus, and BMV denotes brome mosaic virus.

Cationic compounds of formula (I) are helpful in delivering anionic therapeutics. Without intending to be bound to a particular theory through micelle formation, a therapeutic is internalized within a BAC shell and protected from gastrointestinal degradation with the micelle inclusive of therapeutic having a lower net charge than the internalized therapeutic itself. Conjugation of the polyionic polypeptide chain to the bile salt is made through the hydroxyl groups in the 3, 7, or 12 positions of a bile acid steroid nucleus, or on the 24-carboxyl group of the bile acid or aforementioned bile acid derivative or via other substituent types or locations on the derivative to yield an amidyl linkage to X or Z peptides. Preferably, an amidyl linkage is present between R and the peptide tail, although it is appreciated that ether, ester, sulfonyl, of other hydrolyzable bonds are also operative herein and formed through the bile acid carboxylic acid moiety, a hydroxyl moiety extending from the tetracyclic core, and an additional core substituent or ethylenic unsaturation.

Anionic small molecule therapeutics represent a broad class of pharmaceuticals that are exemplary of the benefits of the present invention. By way of example, a carboxyl group imparts a negative charge that lessens bioavailability leading to higher dosing and side effects. Acid containing drugs containing carboxylic, benzoic groups found in existing drugs illustratively include 1-dopa, angiotensin-converting enzyme inhibitors such as: benazeprilat, captoprilat, enalaprilat, fosinoprilat, lisinoprilat, perindoprilat, ouinaprilat, ramiprilat, spiraprilat, trandolaprilat and moexiprilat; cephalosporin antibiotics such as: cefaclor, cefadroxil, cefamandole, cefatrizine, cefazedone, cefazuflur, cefazolin, cefbuperazone, cefclidine, cefepime, cefetecol, cefixime, cefluprenam, cefmenoxime, cefmetazole, cefodizime, cefonicid, cefoperazone, ceforanide, cefotaxime, cefotefan, cefotiam, cefoxitin, cefpimizole, cefpirome, cefoselis, cefozopran, cefpirome, cefquinome, cefpodoxime, cefroxadine, cefsulodin, cefpiramide, ceflazidime, ceftezole, ceftizoxime, ceftriaxone, cefuroxime, cephacetrile, cephalexin, cephaloglycin, cephaloridine, cephalosporin, cephanone, cephradine, and latamoxef; penicillins such as amoxycillin, ampicillin, apalcillin, azidocillin, azlocillin, benzylpencillin, carbenicillin, carfecillin, carindacillin, cloxacillin, cyclacillin, dicloxacillin, epicillin, flucloxacillin, hetacillin, methicillin, mezlocillin, nafcillin, oxacillin, phenethicillin, piperacillin, sulbenicllin, temocillin, and ticarcillin; carbapenems a class of beta-lactam antibiotics such as: imipenem, meropenem, ertapenem, faropenem, doripenem, danipenem/betamipron; tazobactam which inhibits the action of bacterial beta-lactamases extending the spectrum of beta-lactam antibiotics; thrombin inhibitors such as argatroban, melagatran, and napsagatran; influenza neuraminidase inhibitors such as zanamivir, peramivir and oseltamivir; non-steroidal anti-inflammatory agents such as acametacin, alclofenac, alminoprofen, aspirin acetylsalicylic acid), 4-biphenylacetic acid, bucloxic acid, carprofen, cinchofen, cinmetacin, clometacin, clonixin, diclenofac, diflunisal, etodolac, fenbufen, fenclofenac, fenclosic acid, fenoprofen, ferobufen, flufenamic acid, flufenisal, flurbiprofin, fluprofen, flutiazin, ibufenac, ibuprofen, indomethacin, indoprofen, ketoprofen, ketorolac, lonazolac, loxoprofen, meclofenamic acid, mefenamic acid, 2-(8-methyl-10,11-dihydro-11-oxodibenz[b,f]oxepin-2-yl)propionic acid, naproxen, nifluminic acid, O-(carbamoylphenoxy)acetic acid, oxoprozin, pirprofen, prodolic acid, salicylic acid, salicylsalicylic acid, sulindac, suprofen, tiaprofenic acid, tolfenamic acid, tolmetin and zopemirac; prostaglandins such as ciprostene, 16-deoxy-16-hydroxy-16-vinyl prostaglandin E₂, 6,16-dimethylprostaglandin E₂, epoprostostenol, meteneprost, nileprost, prostacyclin, prostaglandins E₁, E₂, or F_{2α}, and thromboxane A₂; quinolone and fluoroquinolone antibiotics such as: acrosoxacin, cinoxacin, ciprofloxacin, enoxacin, fleroxacin, flumequine, gatifloxacin, gemifloxacin, grepafloxacin, levofloxacin, lomefloxacin, moxifloxacin, naladixic acid, norfloxacin, ofloxacin, oxolinic acid, pefloxacin, pipemidic acid, piromidic acid, prulifloxacin, rufloxacin, rosoxacin, sitafloxacin, sparfloxacin, temafloxacin, and trovafloxacin; other antibiotics such as aztreonam, imipenem, meropenem, and related carbopenem antibiotics; anticonvulsants such as clorazepate, gabapentin and valproic acid; meglitinides such as: nateglinide, repaglinide, and mitiglinide; diuretics such as furosemide; statins such as: atorvastatin, cerivastatin, fluvastatin, lovastatin acid, mevastatin acid, pitavastatin, pravastatin acid, rosuvastatin and simvastatin; antihypertensive such as: hydralazine; antimetabolites such as: pemetrexed; calcium channel blockers such as nicardipine; bisphosphonates such as: pamidronic acid, alendronic acid, ibandronic acid, risedronic acid, zoledronic acid etidronic acid, clodronic acid and tiludronic acid; immunosuppressive agents such as: mycophenolic acid; anticancer agents such as: etoposide phosphate, melphalan, methotrexate and pemetrexed; angiotensin II receptor antagonists such as: candesartan, telmisartan and valsartan; antifibrinolytic agents like aminocaproic acid; acetohydroxamic acid, which is prescribed to decrease urinary ammonia, and may help antibiotics to work or help with other kidney stone treatments; verteporfin, which is a medication used as photosensitizer for photodynamic treatment to eliminate the abnormal blood vessels in the eye; Liothyronine which is a thyroid hormone drug used to treat hypothyroidism; cromolyn used in an oral form to treat mastocytosis, dermatographic urticaria and ulcerative colitis; penicillamine which is used as a form of immunosuppression to treat rheumatoid arthritis and as a chelating agent in the treatment of Wilson's disease; dimercaptosuccinic acid used as a heavy metal chelating agent; ethacrynic acid which is used as a loop diuretic medication; montelukast which is an oral leukotriene receptor antagonist (LTRA) for the maintenance treatment of asthma and to relieve symptoms of seasonal allergies; misoprostol acid which is used for the treatment and prevention of stomach ulcers, to induce labor and as an abortifacient.

By way of a second example, a phosphonate or phosphate group imparts a negative charge that lessens bioavailability leading to higher dosing and side effects. Phosphate and phosphonate containing existing drugs illustratively include: antiviral compounds including adefovir, cidofovir, cyclic cidofovir, foscarnet, and tenofovir,

Anionic amino acid chains operative herein as the Z moiety in formula (I) illustratively include a single aspartic acid or glutamic acid residue, a 2-50 residue oligopeptide that contains at least 20 residue percent aspartic acid or at least 20 residue percent glutamic acid, or a combination thereof. Preferably, more than 30 residue percent aspartic acid, more than 30 residue percent glutamic acid, or combination thereof is present. Most preferably Z is less than 25 total residues in length and more than 35 residue percent aspartic acid, more than 35 residue percent glutamic acid, or combination thereof. Specific examples of anionic Z moieties effective in internalizing a bile acid or derivative thereof moiety R and a co-administered therapeutic include wholly aspartic acid or wholly glutamic acid oligopeptides having a length of from 1 to 12 residues, and synthetic residues (EANA)ₙE where n is an integer 2-5 (SEQ ID NOS. 19-22, respectively). Anionic compounds of formula (I) are helpful in delivering cationic therapeutics through micelle formation, with the micelle having a lower net charge than the internalized therapeutic.

The organic small molecules positively charged therapeutics are principally delivered as salts and in particular hydrochloride salts and at physiological pH are present as cations, with the charge tending to decrease bioavailability even while increasing chemical solubility. Cationic small molecule therapeutics suitable for the delivery according to the present invention illustratively include: antacids such as magnesium hydroxide, aluminum hydroxide, and calcium carbonate; iron products such as ferrous sulfate, ferrous gluconate, ferrous fumarate, and iron-polysaccharide complex; mineral containing multivitamins; antireflux agents such as sucralfate; potassium-sparing diuretics such as amiloride, and triamterene; cardiac glycosides such as digoxin; opioid analgesics such as morphine; antiarrhythmics such as procainamide, quinidine, and quinine; histamine (H2) blockers such as ranitidine; antimicrobials such as trimethoprim, vancomycin, and gentamycin; local anesthetics such as tetracaine, and lidocaine; antipsychotics such as chlorpromazine; beta-blockers such as propranolol; antivirals such as amantadine; and sympathomimetic agents such as pseudoephedrine; neuromuscular-blocking drugs or muscle relaxants such as: atracurium, mivacurium and cisatracurium as well as the smooth muscle relaxant dicyclomine; selective alpha 1 antagonists such as: alfuzosin and terazosin; calcium channel blockers such as: amlodipine, verapamil and gallopamil; endothelin receptor antagonist such as: bosentan; calcimimetic drugs such as: cinacalcet; selective beta1 receptor blockers like metoprolol, propranolol, timolol and atenolol; cholinesterase reactivators such as: pralidoxime; cytotoxic/antitumor antibiotics such as: daunorubicin, doxorubicin, epirubicin, idarubicin, and mitoxantrone; topoisomerase 1 inhibitors such as irinotecan; gonadotropin-releasing hormone agonists such as leuprolide; antihypertensive agents such as: apraclonidine, clonidine, doxazosin, guanethidine, guanfacine, lofexidine, mecamylamine, methyldopa, moxonidine and prazosin; triptan drug like almotriptan, and rizatriptan; blockers of M3 muscarinic acetylcholine receptors such as: darifenacin; the amphetamine-like prescription stimulant methylphenidate; 5-HT4 agonists such as tegaserod; 5-hydroxytryptamine 1B/1D (5-HT 1B/1D) receptor agonists like zolmitriptan; atypical antipsychotics such as: ziprasidone; mesylate, quinazoline alpha blockers such as doxazosin and phenoxybenzamine; drugs used to treat male erectile dysfunction such as sildenafil and the phosphodiesterase type 5 inhibitor vardenafil; small molecule receptor tyrosine kinase inhibitors such as sunitinib; antiretroviral drugs such as nelfinavir; antidepressants of the selective serotonin reuptake inhibitor (SSRI) type such as sertraline; anti-diabetic drugs from the biguanide class such as metformin; serotonin 5-HT3 receptor antagonists like granisetron; orally administered antimalarial drugs used as a prophylaxis such as mefloquine; short-acting nonbenzodiazepine hypnotics such as zolpidem; inhibitor of phosphodiesterase-3 such as anagrelide; alpha-sympathomimetic drugs like midodrine; inhibitors of monoamine oxidase such as selegiline; ergoline-based dopamine receptor agonists like pergolide; glycopeptide antibiotics such as: vancomycin, telavancin and oritavancin; serotonin-norepinephrine reuptake inhibitors such as venlafaxine; broad spectrum glycylcycline antibiotics like tigecycline; selective estrogen receptor modulators such as: tamoxifen and 4-hydroxytamoxifen; vasodilators such as: hydralazine and dihydralazine; activators of the intracellular receptor class of the peroxisome proliferator-activated receptors such as: pioglitazone.

In use as a therapeutic delivery formulation, an inventive compound (I) is added in solution to the designed therapeutic and uptake levels of the therapeutic simulated by monitoring the partition coefficient of the therapeutic alone and in combination with a compound of formula (I). A partition coefficient monitoring technique is detailed in Example 4. In vivo optimization of the ratio of a therapeutic to compound (I) is determined by varying the ratio and monitoring blood serum levels of the therapeutic as a function of the ratio. Typically the compound (I) is present in a molar ratio of compound (I): therapeutic ranges from 0.005:1 to 500,000:1 and preferably in a ratio of from 0.05 to 50,000:1. More preferably, the molar ratio of compound (I): therapeutic ranges from 0.5:1 to 20,000:1. For a therapeutic, a molar ratio 0.1:1 to 10:1 is most preferred.

Without being intended to be limited to a particular theory, it is believed that one of the mechanisms to permit transport of highly ionized therapeutics through cell membranes requires the pairing of charged therapeutic with a compound of formula (I) having an equal but opposite charge to yield a neutral complex that can passively diffuse through the lipid membrane, synonymously described herein as ion pairing. Single charges are appreciated to often lack sufficient affinity to a therapeutic so as to increase bioavailability, and other mechanisms involving the bile salts interaction may facilitate the increase in bioavailability observed.

In the case of an anionic therapeutic at physiological pH, formulating with a compound (I) that is polycationic serves to enhance bioavailability. While the specific mechanism remains unknown, obtaining a net charge balance between the total quantity of therapeutic and compound (I) at physiological pH represents an initial estimate as to the relative quantity of compound (I) to be used.

The invention involves methods and products for oral, parenteral, mucosal, transdermal, and infusion delivery of therapeutics for both systemic and localized therapy by increasing the efficiency of absorption of a therapeutic complex with a compound of formula (I) into the cells. Cells of a mammalian subject, either intestinal epithelia after oral delivery, or cells in other organs after other forms of inventive delivery, are altered to operatively incorporate a therapeutic. These complexes also optionally are delivered to other organs of the body in a variety of ways, including direct injection or infusion. In a preferred embodiment administration is oral and targeted to transport a therapeutic into intestinal epithelial cells.

The present invention has utility as a treatment for a variety of disease conditions or deficiencies through nucleic acid delivery. These conditions and deficiencies illustratively include: enzyme deficiency, erythropoietin, catalase, endotoxic shock/sepsis, adenosine deaminase for treatment of severe combined immunodeficiency, lipid-binding protein (LBP), purine nucleotide phosphorylase, galactosidase, beta-glucuronidase, antioxidants for cancer, therapy anemia, superoxide dismutase, cancer, differentiated and non-differentiated cell growth, growth factors for use in wound healing, induction of red blood cell formation and the like, α-interferon, β-interferon, epidermal growth factor, granulocyte colony stimulating factor (G-CSF), alpha-IL1, gamma-interferon, phenylalanine ammonia lyase, transforming growth factor, arginase, erythropoietin, L-asparaginase, thrombopoietin, uricase, insulin-like growth factor-1, insulin, human growth hormone, monoclonal antibodies, tissue necrosis factor, cardiovascular disease, diabetes, tissue plasminogen activator, urokinase (native or chimeric), glucagon, α₁-antitrypsin, insulinotrophic hormone, clotting disorders, antithrombin-III, other proteases or protease inhibitors, clotting factors VIII, VIIIa, IX, IXa, X, Xa, prothrombin, thrombin, clotting factors VII, VIIa, XIII, XIIIa, XI, XIa, XII, XIIa, V, Va, fibrinogen, fibrin, von Willebrand's factor, TAFI, platelet procoagulant and anticoagulant receptors, apolipoproteins (particularly B-48), circulating scavenger receptor, APO A1 which converts low-density lipoproteins to high-density lipoproteins, gastrointestinal and pancreatic deficiencies, obesity and feeding, pepsin (for esophageal reflux), Ob gene product, cholecystokinin (CCK), trypsin, chymotrypsin, bone diseases, elastase, carboxypeptidase, calcitonin, lactase (for lactose deficiency), PTH-like hormone, sucrase, intrinsic factor (pernicious anemia), myasthenia gravis (acetylcholine receptors), Graves' disease (thyroid-stimulating hormone receptor), organ-specific autoimmune diseases (target of antibody in parentheses), thyroiditis (thyroid, peroxidase), insulin-resistant diabetes with acanthosis nigricans or with ataxia telangiectasia (insulin receptor), allergic rhinitis, asthma (β₂-adrenergic receptors), juvenile insulin-dependent diabetes (insulin, GAD65), pernicious anemia (gastric parietal cells, vitamin B₁₂ binding site of intrinsic factor), Addison's disease (adrenal cells), idiopathic hypoparathyroidism (parathyroid cells), spontaneous infertility (sperm), premature ovarian failure (interstitial cells, corpus luteum cells), pemphigus (intercellular substance of skin and mucosa), bullous pemphigoid (basement membrane zone of skin and mucosa), primary biliary cirrhosis (mitochondria), autoimmune hemolytic anemia (erythrocytes), idiopathic thrombocytopenic purpura (platelet), idiopathic neutropenia (neutrophils), vitiligo (melanocytes), osteosclerosis and Meniere's disease (type II collagen), chronic active hepatitis (nuclei of hepatocytes), systemic autoimmune diseases (defect/organ affected in parentheses), Goodpasture's syndrome (basement membranes), rheumatoid arthritis (γ-globulin, EBV-related antigens, collagen types II and III), Sjogren's syndrome (γ-globulin, SS-A (Ro), SS-B (La), systemic lupus erythematosus (nuclei, double-stranded DNA, single-stranded DNA, Sm ribonucleoprotein, lymphocytes, erythrocytes, neurons, gamma-globulin), soleroderm (nuclei, Scl-70, SS-A (Ro), SS-B (La), centromere, polymyositis (nuclei, Jo-1, PL-7, histadyl-tRNA synthetase, threonyl-tRNA synthetase, PM-1, Mi-2), rheumatic fever (myocardium heart valves), and choroid plexus.

Cells of a mammalian subject, either intestinal epithelia after oral delivery, or cells in other organs after other forms of inventive delivery, are altered to operatively incorporate a gene which expresses a protein, which is secreted directly into the organ and/or bloodstream to provide a therapeutic effect. The use of naked nucleic acid protected by complexation with adsorption and/or internalization factors avoids the complications associated with use of viral vectors to accomplish gene therapy. An inventive complex is delivered via the intestinal lumen in a variety of ways, including through timed-release capsules, such as those detailed in U.S. Pat. No. 4,976,949, thereby obtaining a simple, noninvasive method of gene delivery. These complexes also optionally are delivered to other organs of the body in a variety of ways, including direct injection or infusion.

After oral delivery the transformed intestinal epithelial cells provide short or long term therapies for diseases associated with a deficiency in a particular protein or which are amenable to treatment or palliation by over expression of a protein including metabolic disorders, endocrine disorders, circulatory disorders, coagulation disorders, cancer, and gastrointestinal disease.

An inventive conjugating agent has the general formula:
A-R₁-Q-Y-Z (II)

where R₁ is a cholesterol derivative; a C₈-C₂₄ alkyl; C₈-C₂₄ heteroatom substituted alkyl wherein the heteroatom is O, N or S; where A is a nullity (the absence of an additional group), C₀-C₄ alkyl-hydroxy, -substituted amino, -quaternary amino, -sulfonate, -phosphonate, and -carboxylate; and targeting ligand; where the targeting ligand includes amino acids, hormones, antibodies, cell adhesion molecules, folate, polypeptides, vitamins, saccharides, transferrin, drugs, and neurotransmitters; where Q is sulfur, a secondary amine, or oxygen; where Y is a linker peptide having a negative, neutral, or positive charge; and where Z is a polyionic peptide as specified with respect to formula (I). Specific examples of inventive R₁ cholesterol derivatives illustratively include cholestanol, coprostanol, cholic acid, glycocholic acid, ursoldeoxycholic acid, chenodeoxycholic acid, desoxycholic acid, glycochenodeoxycholic acid, taurocholic acid, and taurochenodeoxycholic acid. Specific examples of C₈-C₂₄ alkyls are 13-hydroxyl tridecanoic acid; 1,12 dodecane diol; and 1,12 dodecanediame.

A peptide linker sequence Y is preferably employed to separate A-R₁-Q and the polyionic peptide sequence Z that interacts with the nucleic acid by a distance sufficient to ensure that the cholesterol derivative is sterically accessible and that the polyionic peptide Z folds into its secondary and tertiary structures. Such a peptide linker sequence Y is incorporated into an inventive compound using standard techniques well known in the art. Suitable peptide linker sequences Y are chosen based on the following factors: (1) their ability to adopt a flexible extended conformation; (2) their inability to adopt a secondary structure that could interact with functional epitopes of the inventive compound; and (3) the lack of hydrophobic or charged residues that might react with the polyionic peptide functional epitopes. Preferred peptide linker sequences contain Gly, Asn and Ser residues. Other near-neutral amino acids, such as Thr and Ala, also are operative in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. The linker sequence may be from 0 to about 50 amino acids in length. A peptide linker sequence Y is not required when the polyionic peptide Z has non-essential N-terminal amino acid regions that can be used to separate the functional domains and prevent steric interference.

A polyionic peptide Z according to formula (II) is the same as defined with respect to formula (I).

Proteins usefully expressed by genes transfected with the composition of formula (II) through the administration of the present invention illustratively include proteases, pituitary hormones, protease inhibitors, growth factors, cytokines, somatomedians, chemokines, immunoglobulins, gonadotrophins, interleukins, chemotactins, interferons, and lipid-binding proteins, specific examples of which illustratively include insulin, interferon-α2B, human growth hormone (hGH), transforming growth factor (TGF), erythropoietin (EPO), ciliary neurite transforming factor (CNTF), clotting factor VIII, insulin-like growth factor-1 (IGF-1), bovine growth hormone (BGH), granulocyte macrophage colony stimulating factor (GM-CSF), platelet derived growth factor (PDGF), interferon-α2A, clotting factor VIII, brain-derived neurite factor (BDNF), thrombopoietin (TPO), insulintropin, tissue plasminogen activator (tPA), IL-1, IL-2, urokinase, IL-1 RA, streptolcinase, superoxide dismutase (SOD), adenosine deamidase, catalase, calcitonin, arginase, fibroblast growth factor (FGF) (acidic or basic), neurite growth factor (NGF), phenylalanine ammonia lyase, granulocyte colony stimulating γ-interferon factor (G-CSF), L-asparaginase, pepsin, uricase, trypsin, chymotrypsin, elastase, carboxypeptidase, lactase, sucrase, intrinsic factor parathyroid hormone (PTH)-like hormone, calcitonin, Ob gene product, cholecystokinin (CCK), glucagon, glucagon-like-peptide I (GLP-1), and insulinotrophic hormone.

The conjugate agent A-R₁-Q-Y-Z is preferably a bile acid conjugated with a polycationic peptide linked to the bile acid steroid backbone. The bile acid moiety acts to target the conjugate to bile acid transporters in the lumen of the intestine and assist in the cellular internalization of the complex. Short polycation peptides rich in arginine or lysine, such as a six amino acid residue or longer chain, provide multiple functions illustratively including: a) having an affinity for nucleic acid, b) act condensing agent, c) protect the nucleic acid from nuclease activity and d) assist in cellular internalization of the complex.

Conjugation of the polyionic polypeptide chain to the bile salt is made through the hydroxyl groups in the 3, 7, or 12 positions of a bile acid steroid nucleus, or on the 24-carboxyl group of the bile acids.

Composition of the polyionic peptide nucleic acid condensing moiety is altered to vary the affinity to the nucleic acid, providing changes in the rate of intracellular decondensing of the nucleic acid. These alterations are known to one skilled in the art.

Alternatively, A is a targeting ligand. A targeting ligand according to the present invention is any molecule which binds to specific types of cells. A targeting ligand may be any type of molecule having a corresponding cellular receptor. Ideally, a targeting ligand is recognized by a cellular receptor and expressed only on a specific type of cell thereby affording selectivity. Examples of targeting ligands which are operative herein include amino acids, hormones, antibodies, cell adhesion molecules, folate, polypeptides, vitamins, saccharides, transferrin, drugs, and neurotransmitters. The 12-residue membrane-translocating peptide (Ala-Ala-Val-Leu-Leu-Pro-Val-Leu-Leu-Ala-Ala-Pro) (SEQ ID NO: 23) is exemplary of a target ligand form of A. The targeting ligand operative to either target, increase adsorption, internalization or nuclear localization of the inventive compound.

In another embodiment, the selected nucleic acid is, or encodes, an RNA molecule comprising an antisense which blocks expression of a gene, e.g., in a tumor cell. By blocking expression of this selected gene, inhibition of growth is observed for the tumor.

It is appreciated that a nucleic acid delivered according to the present invention includes a ribozyme. A ribozyme is a catalytic RNA molecule that cleaves other RNA molecules such as mRNA transcripts in a cell. Common targets include RNAs having GUC or GUA subsequences. For example, hairpin ribozymes typically cleave one of two target sequences. GUC hairpin ribozymes cleave an RNA target sequence consisting of NNNBCN*G UCNNNNNN (SEQ ID NO: 24) where N*G is the cleavage site, B is any of G, U or C, and where N is any of G, U, C, or A. GUA ribozymes typically cleave an RNA target sequence consisting of NNNNN*GUANNNNNNNN (SEQ ID NO: 25) where N*G is the cleavage site and where N is any of G, U, C, or A. See, De Young et al. (1995) Biochemistry 34: 15785-15791. Ribozymes delivered optionally have non-standard ribonucleotide bases, or deoxyribonucleotide bases, which can stabilize the ribozyme and make the ribozyme resistant to RNase enzymes. Alternatively, the ribozyme is modified to a phosphothio analog, thereby rendering the ribozyme resistant to endonuclease activity.

According to the present invention, a systemic effect is achieved by transducing cells with genes that express proteins that are secreted into the circulatory system and therefore provide a systemic effect. As is known to one skilled in the art, deleting from a gene pro-protein sequences or transporter sequences tends to restrict a protein to an intracellular effect. Additionally, according to the present invention gene expression and therefore therapeutic effect is regulated through administration. In an embodiment where intestinal epithelial cells are transduced, these cells are sloughed from the lumen every several days thereby affording only transient gene expression. Conversely, the transduction of cells with less turnover than intestinal epithelial cells affords longer gene expression periods.

The compounds of the present invention can be administered to a patient either alone or a part of a pharmaceutical composition. The compositions can be administered to patients either orally, rectally, parenterally (intravenously, intramuscularly, or subcutaneously), intracistemally, intravaginally, intreperitoneally, intravesically, locally (powders, ointments, or drops), or as a buccal or nasal spray.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol or a suppository wax, which are solid at ordinary temperatures but liquid at body temperature and therefore melt in the rectum or vaginal cavity and release the active component.

Dosage forms for topical administration of a compound of this invention include ointments, powders, sprays, and inhalants. The active component is admixed under sterile conditions with a physiologically acceptable carrier and preservatives, buffers, or propellants as may be required. Ophthalmic formulations, eye ointments, powders, and solution are also contemplated as being within the scope of this invention.

An inventive compound is also delivered in conjunction with an active therapeutic compound, a pharmaceutically acceptable salt, ester, amide or prodrug thereof. The therapeutic compounds are listed above in anionic and cationic forms and illustratively are active as antibiotic, a gamma or beta radiation emitting species, an anti-inflammatory, an antitumoral, an antiviral, an antibody, a hormone, an enzyme, and antigenic peptide or protein.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like, (See, for example, S. M. Barge et al., “Pharmaceutical Salts,” J. Pharm. Sci., 1977, 66:1-19.)

The present invention is further detailed with respect to the following non-limiting examples. While the following examples illustrate the present invention through exemplary anionic therapeutic-cationic inventive compound delivery combinations, analogs are found among cationic therapeutic-anionic inventive compound pairings. The examples presented below are intended to illustrate particular embodiments of the invention and are not intended to limit the scope of the specification, including the claims.

### EXAMPLES

### Example 1

### Method for Synthesis of Bile Acid Conjugates (BAC)

BAC is synthesized by solid phase chemistry on a peptide synthesizer. A six L-arginine peptide is first synthesized on the resin bed using standard 9-fluorenylmethoxycarbonyl (FMOC) chemistry. To attach the bile acid salt, an excess of chendoxycholic acid is added to the resin and allowed to react with the immobilized peptide. After conjugation, the N-hexapeptide (A6 motif in Table 1) chenoxycholamide BAC is cleaved from the resin and purified to greater than 95% purity by HPLC.

### Example 2

### Measurement of the Critical Micellar Concentration for Bile Acid Conjugates (BAC) of Compound I

The critical micellar concentration (CMC) of the BACs is determined using a dye solubilization method which monitored the partitioning of the dye into micelle as a function of the BAC concentration (Wang et al. Biomacromolecules 2002; 3(6):1197-207). Briefly, serial dilutions of a 7 mg/ml solution of the BACs are made in 50 mM Tris buffer pH 8.0. 10 ul of the dye 1,6-diphenyl-1,3,5-hexatriene (DPH, Sigma) (0.4 mM in methanol) is added to each ml. After overnight RT incubation in the dark, the absorbance of the samples at 356 nm is recorded. Linear regression of the data points above baseline was performed and the calculated CMC derived from the x intercept. The results for thirteen different BACs are shown in Table 1 based on a chendoxycholamide of the detailed motifs.

TABLE 1
BAC CMC Measurement

| | | | |
|---|---|---|---|
| | Peptide, | | Calculated CMC |
| | Z of formula | | concentration |
| BAC abbreviation | (I)= | M.W. | (mM)¹ |

Motif A-(R-)

| | | | |
|---|---|---|---|
| A3 | -RRR | 861.14 | 3.5 |
| A4 | -RRRR | 1017.3 | 1.8 |
| A6 | -RRRRRR | 1329.7 | 2.6 |
| Motif B-(R-R-R-A-) | | | |
| B7 | -RRRARRR | 1400.8 | >5.0* |
| B11 | -RRRARRRARRR | 1940.4 | 1.9 |
| | (SEQ ID NO.26) | | |
| Motif C-(R-R-A-) | | | |
| C5 | -RRARR | 1088.4 | >6.4* |
| C8 | -RRARRARR | 1471.8 | 4.4 |
| C11 | -RRARRARRARR | 1855.3 | 1.2 |
| | (SEQ ID NO.27) | | |
| Motif D-(R-A-) | | | |
| D3 | -RAR | 776.03 | >9.0* |
| D9 | -RARARARAR | 1457.8 | 1.7 |
| D11 | -RARARARARAR | 1685 | 2.5 |
| | (SEQ ID NO.28) | | |
| Motif E-(R-A-W-A-) | | | |
| E9 | -RAWARAWAR | 1517.8 | 1.2 |
| E13 | -RAWARAWARAWAR | 2002.3 | 0.8 |
| | (SEQ ID NO.29) | | |

*The CMC for three BACs D3, C5, and B7 could not be determined, and are greater than 7 mg/ml (5-9 mM for the three BACs), the highest concentration tested. The experimental range reflects the range of CMCs calculated by varying the range of data points included in the linear regression.

### Example 3

### Preparation of BAC/Drug Mixture

Typically a volume of the drug at the desired concentration in 10 mM Tris Buffer (pH 7.4) was added to a quantity of BAC solid to yield equal molar concentrations of the drug and BAC in solution. Mass spec analysis shows that the major ionic species in the BAC has a charge of +3 per BAC_A6 (N-A6 motif of chenoxycholamide) molecule (MW=1329) under neutral pH 7.0. Thus, charge neutralization of the anionic therapeutic is expected.

### Example 4

### Alteration of the Octanol/Water Partition Coefficient for Fluorescein and Carboxylfluorescein by Various BACs of Formula I

The interaction of cationic BACs with various therapeutics can be shown by determining the alterations in the partition coefficient between hydrophilic and hydrophobic liquid phases for the therapeutic. In particular, changes in the lipid solubility of the anionic fluorescent dyes carboxyfluorescein and fluorescein as model drugs are measured by dye partitioning between octanol and aqueous buffer at different pHs to determine alterations in the solubility behavior relevant to the permeability of cell membranes in vivo.

Octanol/buffer partition ratios: The ratio of concentrations of a given solute in equilibrium distribution between two immiscible solvents is termed the partition coefficient. This expression properly refers only to the distribution of a single molecular species between the two phases. Both solutes studied exist as a mixture of ionized forms within the pH range tested. Total dye concentration in the two solvent phases is measured without correction for ionization or self-association and the recommended term, “partition ratio”, is used to refer to these uncorrected distributions.

Carboxyfluorescein or sodium fluorescein (both from Sigma Chemical Co., St. Louis) is dissolved at concentrations of 10-3 M or 10-5 M in octanol-saturated 0.01 M Tris buffer at different pH values between 6.40 and 8.03. The aqueous dye solutions are equilibrated with equal volumes of buffer-saturated octanol by 100 inversions during 5 minutes, and the phases are separated by centrifugation. Dye concentrations are measured spectrofluorophotometrically at excitation and emission wavelengths of 485 and 512 nm, respectively, with an Aminco-Bowman Ratio Spectrofluorophotometer.

Two methods are used to calculate partition ratios. In the first, the dye concentration in the aqueous phase is measured before (Cb) and after (Ca) partitioning and the ratio (P.R.) was obtained from the equation:
P.R.=C_{oct}/C_{aq}=(C_{b}−Cₐ)Cₙ,

where C_{oct} and C_{aq} are the dye concentration in the octanol and aqueous phases, respectively after equilibration. In the second method, concentration in the octanol phase is determined by extracting the dye from octanol with an equal volume 0.1 N NaOH. Concentration in the octanol phase could not be measured directly because of the marked loss of fluorescence of both dyes in this solvent. A single base extraction of the octanol recovered 99% of the dissolved fluorescein and 99.9% of the dissolved carboxyfluorescein. Therefore, dye concentration in the first base extract is accepted as the concentration in the octanol phase and calculated the partition ratio (P.R.) from the equation:
P.R.=C_{oct}/(C_{b}−C_{oct})

Both methods for determining the partition ratio gave similar results when more than 10% of the initial dye concentration is removed from the aqueous phase by partitioning. When very small quantities of dye are removed, measurement of concentration differences in the aqueous phase before and after partitioning became unreliable, and only the second method is used to calculate the ratio.

Addition of the BAC compounds of Formula I increased the partitioning of the fluorescent molecules into the octanol organic phase. Likewise fluorescent-labeled peptides and oligonucleotides increased their hydrophobicity when ion paired with BAC compounds.

Representative drugs which are fluorescent and can be measured directly in phase partition experiments with BACs are provided in Table 2 in the presence of N-A6 chenoxycholamide (bile acid amidyl-A6).

TABLE 2
Representative drugs which are fluorescent and can be
measured directly in phase partition experiments with BAC

| Drug | Class | λex | λem |
|---|---|---|---|
| Doxorubicin | anthracycline antitumor | 475 | 551 |
| | | 500 | 555,590 |
| | | 480 | 559,593 |
| Daunomycin | anthracycline antitumor | 480 | 560,592 |
| 20-S-Camptothecin | Camptothecins alkaloid antitumor | 370 | 432 |
| 21-lactam-S-Camptothecin | Camptothecins alkaloid antitumor | 368 | 440 |
| 10,11-(methylene dioxy) | Camptothecins alkaloid antitumor | 384 | 402,417 |
| Camptothecin | | | |
| Camptothecin 11 | Camptothecins alkaloid antitumor | 370 | 429 |
| SN-38 | Camptothecins alkaloid antitumor | 378 | 525 |
| Topotecan | Camptothecins alkaloid antitumor | 381 | 515 |
| | topoisomerase inhibitor | 400 | 540 |
| Tricyclic ketone-Camptothecin | Camptothecins alkaloid antitumor | 335 | 429 |
| N,N-Dimethyltryptainine | hallucinogenic | 281 | 361 |
| Mescaline hemisulfate | hallucinogenic | 331 | 381 |
| Ibogaine hydrochloride | hallucinogenic | 300 | 364 |
| Mescaline hydrochloride | hallucinogenic | 330 | 380 |
| Propranolol | quinoline derivatives nonselective β- | 295 | 330 |
| | adrenergic blocker | | |
| 5-aminothiobendazole | Benzimidazole analogs | 260* | 520 |
| | anthelmintic and fungicide | | |
| Protoporphyrin IX | Metabolite of PDT drug α- | 405 | 635 |
| | aminolevulinic acid (ALA) | | |
| Morphine | Opiate agonist | 280* | 335 |
| Tetracycline | Antibiotic | 385* | 500* |
| Salicylic Acid | nonsteroidal anti-inflammatory | 290 | 404 |
| Acetyl salicylic acid (aspirin) | nonsteroidal anti-inflammatory | 310* | >389* |
| N-acetyl 5-aminosalicylic acid | nonsteroidal anti-inflammatory | 315* | 430* |
| Piroxicam | nonsteroidal anti-inflammatory | 330 | 460* |
| Naproxen | nonsteroidal anti-inflammatory | 330 | 553 |
| Flufenamic acid | nonsteroidal anti-inflammatory | 350* | 420 |
| Mefenamic acid | nonsteroidal anti-inflammatory | 350* | 465 |
| Meclofenamic acid | nonsteroidal anti-inflammatory | 350* | 397 |
| Fenoldoparn | dopamine receptor agonist | 336* | >400* |
| Amiloride | diuretic | 382 | 416 |
| Triamterene | diuretic | 370 | 434 |
| Bendroflumethiazide | diuretic | 272 | 381 |
| Bumetamide | diuretic | 350 | 428 |
| Furosemide | diuretic | 365 | 410 |
| Triamterene | diuretic | 372 | 436 |
| Piretamide | diuretic | 348 | 437 |
| Flecainide | antiarrhythmatic | 304 | 362 |
| Quinidine | Alkaloid antiarrhythmatic | 363* | 440* |
| Quinine | Alkaloid antimalarial | 335 | 380 |

### Example 5

### Partitioning of BAC Between Buffer and Octanol

Partitioning of the fluorescent compound carboxyfluorescein by a BAC of Formula I into the octanol phase of an octanol/buffer portioning system for a series of BAC compounds is determined following standard methods. An aqueous buffered solution containing 4 nmoles of carboxyfluorescein and 281 nmoles BAC is mixed with an equal volume (475 μl) of buffer saturated 1-Octanol. The mixture is vortexed for 5 mins and the incubated at room temperature for 3 hours. The solution is centrifuged to facilitate complete phase separation and a 100 μl of aqueous material is withdrawn for fluorescent measurement. The results of such a study are presented in Table 3, where the alphanumeric references correspond to those detailed in Table 1.

TABLE 3
Octanol partitioning of Carboxyfluorescein mixed
with different BAC analogs

| | Fluorescence Units | LogD |
|---|---|---|
| Start | 80425 | |
| Water | 78614 | −1.638 |
| A3 | 54209 | −0.316 |
| A4 | 15385 | 0.626 |
| B7 | 58391 | −0.423 |
| B11 | 46 | 3.242 |
| C5 | 67644 | −0.724 |
| C8 | 11110 | 0.795 |
| C11 | 45 | 3.252 |
| D3 | 34124 | 0.133 |
| D9 | 53338 | −0.294 |
| D11 | 71839 | −0.923 |
| E9 | 33143 | 0.152 |
| E13 | 32 | 3.400 |
| CD | 85927 | |

### Example 6

### Construction of Expression Plasmids

An expression plasmid using an optimized Cytomegalovirus (CMV) early promoter-enhancer to drive transgene expression (pCF1) is utilized. For cell localization and transfection expression experiments the firefly luciferase cDNA is used as a reporter transgene (pCF1-luc). Plasmid backbones which are chosen to contain the origin of replication and an antibiotic resistance gene or other selectable marker allowing the growth and maintenance of the plasmid in its bacterial host. Other attributes to facilitate the production of plasmid DNA are: 1) the ability of the plasmid to produce high copy number in the host bacteria; 2) a eukaryotic transcriptional promoter to initiate the synthesis of mRNA when it enters the target cells; 3) the DNA coding sequence encoding the protein of interest with a translational start codon (ATG) in the sequence for initiation of protein synthesis and a stop codon to terminate translation; and 4) an optional transcription termination sequence.

### Example 7

### Preparation of BAC/pDNA Complexes

Typically an equal volume of BAC solution at 3 mg/ml (2.25 mM) is slowly added with mixing to a pDNA solution at a concentration of 250 μg/ml to form a neutrally charged complex. Mass spec analysis shows that the major ionic species in the BAC_A6 (N-A6 motif of chenoxycbolamide) has a charge of +3 per BAC molecule (MW=1329) at pH 7.0. Assuming a charge of −2 per base pair of the 4550 bp pDNA (MW=3,003,000) (total DNA charge=−9100), a net neutral charge would theoretically require a ratio of about 3033:1 (BAC:pDNA). Physical measurements of the BAC/pDNA complex appear require about 10-fold more BAC to neutralize the pDNA charge (see Table 4). Without intending to be bound to a particular theory, the presence of buffer salts bound to the BAC during synthesis and purification at least partially explains this observation.

TABLE 4

| BAC amount | BAC:pDNA molar | Particle Charge determined by |
|---|---|---|
| μg/ml | ratio | Zeta Potential |
| 50 | 8070:1 | Negative |
| 200 | 32,280:1 | Neutral |
| 2000 | 322,799:1 | Positive |

### Example 8

### BAC Condenses Plasmid DNA

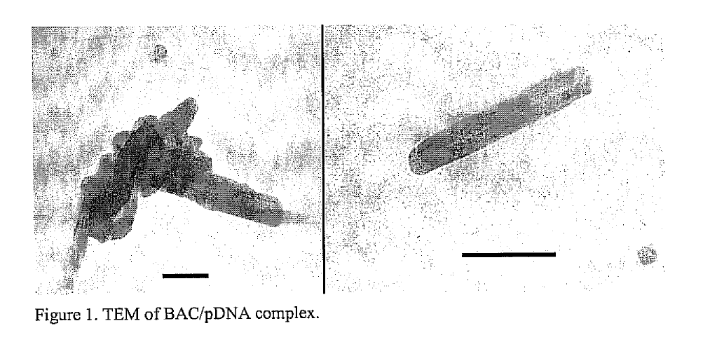

The ability of BAC to condense DNA is determined using transmission electron microscopy (TEM) of a BAC/pDNA particle complex is shown in FIG. 1. The mole ratio of BAC:pDNA is 1581:1, which retained a net negative charge on the particle. The samples after mixing are diluted in water, and a drop applied to the carbon-coated formvar-film grid. The samples are dried and stained with uranyl acetate before viewing. Length bars in FIG. 1 are 100 nM long. Even at this low molar ratio of BAC:pDNA clear rod shaped particles (toroids) are formed.

### Example 9

### BAC Condensed Plasmid DNA Forms Discrete Particles

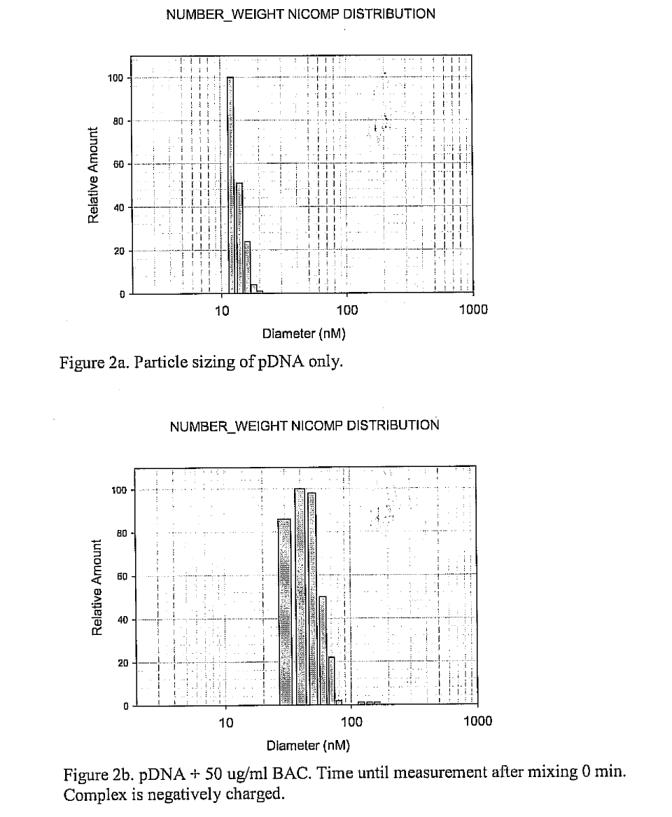

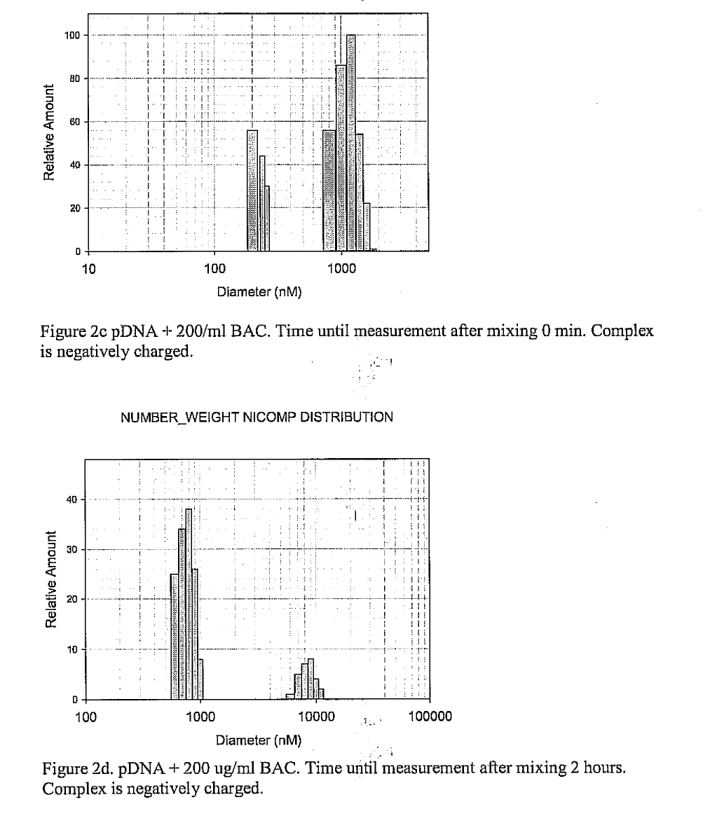

NICOMP particle size analysis is shown in FIG. 2. 1 ml of 14 μg/ml DNA plasmid is mixed with 1 ml of BAC_A6 (N-A6 motif of chenoxycholamide) at the concentrations shown in Table 3. Also shown in Table 3 are the calculated molar ratios and particle charges.

Particle sizes are measured at a specific time after mixing of the pDNA with the BAC.

FIGS. 2(a)-(d) show some representative results of these experiments. Mixtures of pDNA and BAC start forming particles immediately. However, with increasing time these particles appear to aggregate, so that at later times particles larger than 1 μm are observed. FIG. 2(a) is particle sizing for the plasma DNA of Example 2. FIG. 2(b) shows the particle size distribution of 1 ml of 14 microgram per ml DNA plasmid mixed with 1 ml of 50 microgram per ml BAC to yield a negatively charged complex which is sized immediately upon mixing. FIG. 2(c) shows a particle size distribution as per FIG. 2(b) except 200 microgram per ml BAC is introduced instead of 50 microgram per ml BAC to yield a neutrally charged complex. FIG. 2(d) shows the particle size distribution for the experiment according to FIG. 2(c) two hours after mixing.

### Example 10

### BAC Condensed Plasmid DNA Particle Size in Simulated Gastric or Intestinal Fluid

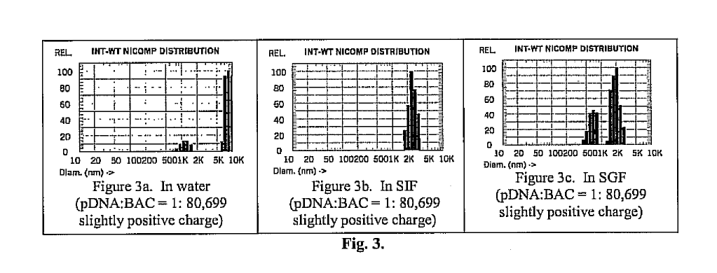

The behavior of the pDNA/BAC complex in buffers with different pHs to simulate gastric fluid (SGF) or intestinal fluid (SIF) are evaluated. pDNA and BAC (BAC:pDNA: =80, 699:1, slightly positive charge) are mixed first in water and then mixed with the same volume of simulated gastric fluid (SGF, 0.1 M HCl, pH 1.2) or simulated intestinal fluid (SIF, 0.05 M phosphate buffer, pH 6.8). Measurements are made after two hours incubation at 37° C. After 2 hours, most of the DNA complexes in water (FIG. 3a) and SIF (FIG. 3b) are observed to be greater than 1 um in size. However, over 30% of DNA complex incubated in SGF (FIG. 3c) is observed to be under 1 μm size. These results indicate that pH has some effect on the physical behavior of BAC/pDNA complexes.

### Example 11

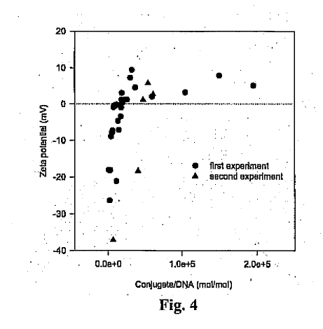

Measurement of the zeta potential (net charge) of the particle complexes are made after mixing BAC/pDNA in water. 800 μl of 11.07 microgram/ml DNA plasmid is mixed with 800 μl of 50 μg/ml BAC (DNA:BAC 1:10,205-negatively charged). 10 μl of 500 μg/ml or 1 mg/ml BAC is added and zeta potential is remeasured in the first experiment (●). 200 microliters of 10 micrograms/ml pDNA is prepared and 2 microliters of 3 mg/ml BAC is added before zeta potential remeasurement in the second experiment (▴). Additional aliquots of BAC are added and the zeta potential remeasured (FIG. 4).

### Example 12

### DNase Protection Assays

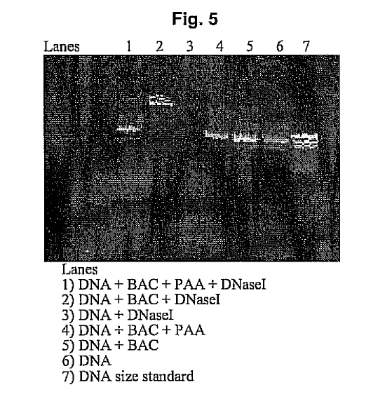

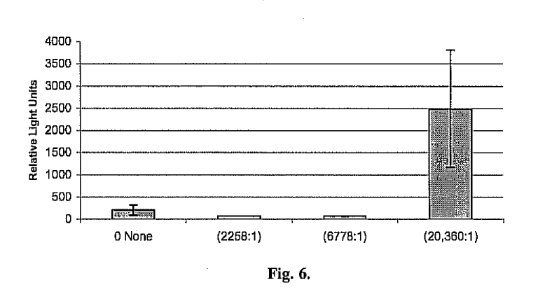

The ability for BAC complex formation to protect DNA from endonuclease activity is examined. We have utilized the ability of polyanions such as polyaspartic acid (PAA) to competitively dissociate the polycations from DNA. Katayose S, Kataoka K. Water-soluble polyion complex associates of DNA and poly(ethylene glycol)-poly(L-lysine) block copolymer. Bioconjug Chem 1997; 8:702-7.
1) The mixture of luciferase BAC/pDNA is stored at room temperature for 2.5 hours (BAC/pDNA: =9038:1).
2) 10 μl of 10 mg/ml PAA or milli-Q water is added to each sample (5 μl of DNA complex mixture) and stored at room temperature for 2 hours.
3) The molar ratio is (DNA:BAC:PAA=1:9038:13,572), the charge ratio (DNA:BAC:PAA=1:3:388), assuming BAC_A6 (N-A6 motif of chenoxycholamide) has 3 positive charges and PAA has 260 negative charges.
4) 5 ul of 100 kunit/ml DNaseI or milli-Q water is added to each sample and those samples are incubated at 37° C. for 15 min and heated at 70° C. for 15 min.
5) 2 ul of 500 mM EDTA and 1 ul of 5% SDS is added to each sample and heated at 55° C. for 3 hours to dissociate the BAC from the pDNA. Samples are then electrophoresed in an agarose gel and stained with ethidium bromide (FIG. 5).

Extremely harsh conditions are required to dissociate BAC from the BAC/pDNA complex (1% SDS at 55° C. for 2 hours). One hour is not enough to reach a plateau dissociation level.

DNaseI together with BAC in the mixture altered the effect the dissociation by SDS (FIG. 5, Lane 2). The DNA sample which is incubated at 37° C. for 15 min with DNaseI showed almost complete fragmentation (FIG. 5, Lane 3). The BAC/pDNA sample incubated in the presence of PAA and DNAseI showed less intact DNA banding (FIG. 5, Lane 1) than BAC/pDNA samples with or without treatment with PAA (FIG. 5, Lanes 4 and 5 respectively).

### Example 13

### In Vitro Transfection Assays

To examine the capability of the BAC/pDNA complexes to transfect cells in vitro, the ability of BAC to transfect Hela tissue cultures using the firefly luciferase gene is examined. In transient transfection assays (24 h), BAC enhanced the transfection efficiency in combination with liposomes (data not shown). In accordance with the present invention, BAC/pDNA is able to transfect Hela cells alone as a single agent (FIG. 6). The charge ratio is approximately neutral. The approximate molar ratios are indicated in parenthesis.

### Example 14

### In Situ, Intestinal Absorption

In order to examine the capability of the BAC/pDNA complex to bind and/or be absorbed onto the intestinal lumen cells the absorption of nicked-radiolabeled pDNA in a single pass perfusion experiment is tested. Using isolated ileal or jejunal segments radio-labeled BAC/pDNA mixtures mixture is mixed with HEPES buffer, pH 7.4, and perused at jejunal (filled symbols) and ileal (open symbols) sites. After 30 min of steady state, sample fractions are collected and the absorbed fraction of BAC/pDNA calculated. BAC/pDNA mixtures used are pDNA:BAC 1:27,000 neutrally charged and 1:54,000 molar ratios positively charged. Uncomplexed pDNA is used as a control.

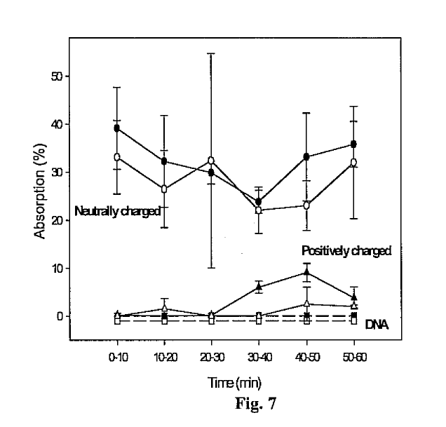

Between 20-40% of the “neutrally charged” BAC/pDNA complex is absorbed in a single pass of the intestine as shown in FIG. 7. As a result, it is appreciated that this complex should be completely absorbed after oral dosing.

### Example 15

### In Situ Intestinal Absorption for Translocating Peptide BAC

The 12-residue membrane translocating peptide (SEQ ID NO: 23) is coupled to bile acid by way of a condensation reaction involving the three alpha hydroxyl group to form an ether linkage there between. The complexes as detailed in Example 10 are recreated with this membrane translocating BAC yielding comparable results to those depicted in FIG. 7.

### Example 16

### Intestinal Administration of an Anionic Therapeutic Enalaprilat

Enalaprilat is administered via an open gut injection in which the therapeutic and N-A6 motif chenoxycholamide BAC absorption enhancer is injected directly into the duodenum of the intestine. Enalaprilat has a molecular weight of 348.4.

As an anionic molecule enalaprilat contains two carboxyl groups that are ionized at physiological pH in solution. Physical measurements of the BAC neutralization of anionic materials preferably using a stoichiometric excess of BAC (on a charge basis) to neutralize the charge associated with co-mixed enalaprilat. Without intending to be bound to a particular theory, the presence of buffer salts bound to the BAC during synthesis and purification at least partially explains this observation.

### Example 17

### Octanol Partitioning of Enalaprilat as a Function of BAC Concentration

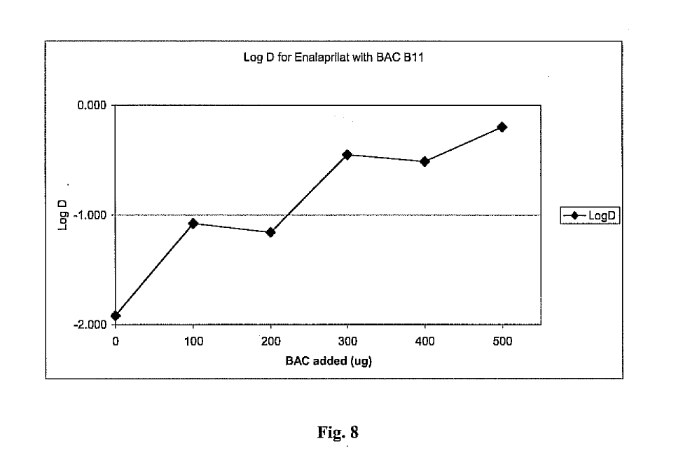

Partitioning of the drug enalaprilat by increasing concentrations of the BAC, (bile acid amidyl B11) into the octanol phase of an octanol/buffer portioning system are summarized below. 0.3 mg/ml (100 microliters) of enalaprilat is mixed with amounts of N-B11-motif chenoxycholamide BAC (bile acid amide of B11 motif per Table 1) ranging from 0 to 160 microliters of 5 mg/ml BAC solution (800 microgram max). To each solution is added 50 microliters buffer 10× (100 mM Tris 7.4), followed by the addition of 500 microliters buffer saturated 1-octanol. The resulting two-phase mixture is vortexed 2 hours, centrifuged 5 minutes at 10,000 rpm, with aqueous diluted to 1 ml. A 20 microliter aliquot is measured as shown in FIG. 8. The experiment is repeated with N-E13 motif ursocholamide (E13 motif per Table 1) and achieved results comparable to those shown in FIG. 8.

### Example 18

### Oral Antibody Delivery as an Example of Protein Uptake

Antibodies can be delivered to the systemic circulation via the oral route. Mouse monoclonal IgG antibodies (100 ug) specific for a synaptic vesicle protein (SV2) are diluted into 0.5 ml PBS or PBS containing 972 μg of BAC_A6 (N-A6 motif of chenoxycholamide). Mice are orally gavaged with the above material, or the antibody in PBS is injected intraperitoneally as a control. At various time points samples of blood are obtained and the titer of the anti-SV2 antibody in the serums were determined by ELISA. Detectable levels of the anti-SV2 antibodies are detectable in the systemic circulation after oral dosing of antibodies combined with the BAC compound.

### Example 19

### Intestinal Administration of Anionic Therapeutic Zanamivir

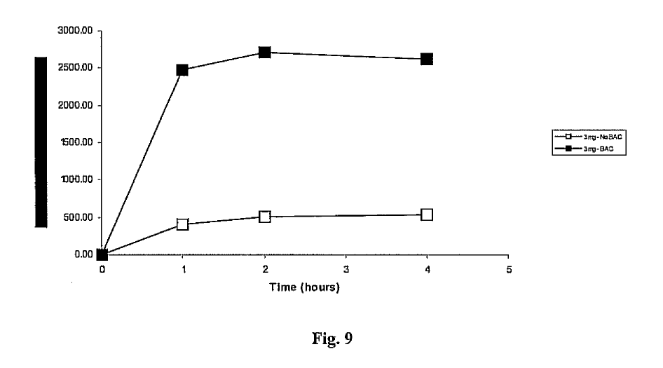

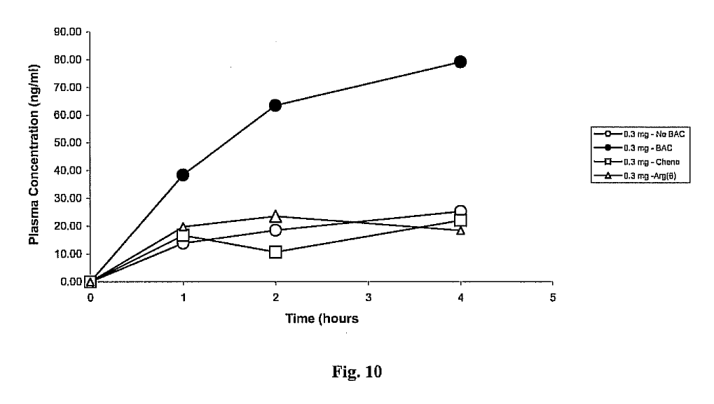

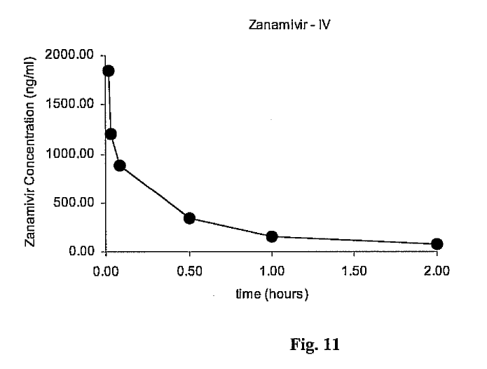

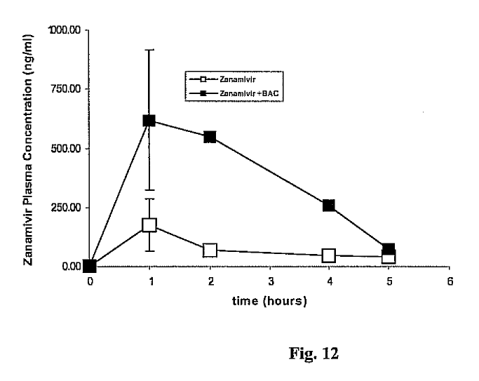

Zanamivir is administered via an open gut injection in which the drug and N-A3 motif chenoxycholamide BAC absorption enhancer (A3 motif per Table 1) is injected directly into the duodenum of the intestine as shown in FIG. 9. Zanamivir has a molecular weight of 332.3. This is compared to the IV dose to determine bioavailability as the fraction absorbed as shown in FIG. 10. As an anionic molecule zanamivir contains one carboxyl group that is ionized at physiological pH in solution. Comparable results are obtained with lithocholic acid bound directly to motif D9 per Table 1, as well as for lithocholic acid amide bound to an intermediate triglycinyl linker (X of formula (I)) that is coupled to motif D3 per Table 1 (lithochol amidyl-GGG-RAR).

### Example 20

### BACs Enhance the Bioavailability of Zanamivir

Groups of mice (8 or 10) are dosed by oral gavage with a solution (0.2 ml) containing zanamivir with or without the prototype BAC analog, BAC_A6 (N-A6 motif of chenoxycholamide), at different dosing levels. The BAC:drug mixtures are incubated for 15-30 minutes at room temperature prior to dosing. At each time point, two mice are sacrificed from each group and plasma is collected by cardiac puncture. Zanamivir concentrations are determined by LC/MS/MS analysis.

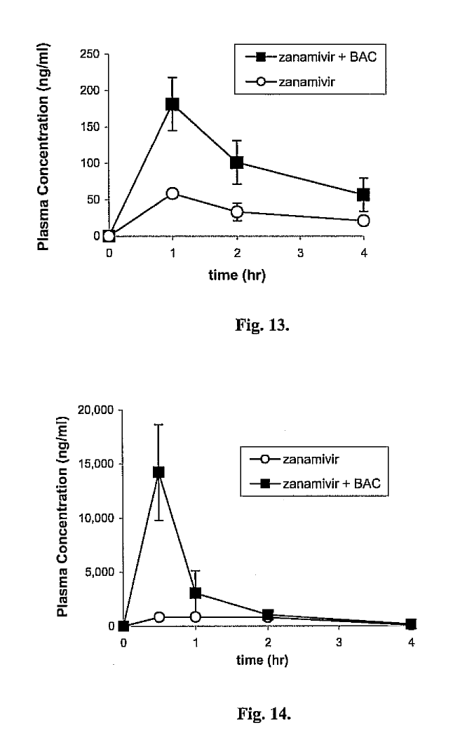

The results of the data are presented in FIGS. 13-16. These examples demonstrate the BAC enhancement of zanamivir absorption in mice when the BAC:zanamivir complex is orally administered by gavage. The effect is evident over at least a 10-fold range in dosage level and is seen in both fed and fasted state. Further, altering the ratio of BAC to drug can significantly alter the extent of absorption.

Oral dosing of zanamivir (4 mg/kg) with and without BAC (12 mg/kg) in fasted mice. Fasted animals are dosed by oral gavage with zanamivir at 4 mg/kg (open circles) or Zanamivir at 4 mg/kg+12 mg/kg BAC-A6 (filled squares) is shown in FIG. 13, with plasma zanamivir levels determined at the times indicated. Each data point represents the average of two mice. Mice dosed with the BAC:zanamivir complex show a >3 fold increase in plasma levels compared to mice dosed with Zanamivir alone. This demonstrates that the BAC enhancement effect can withstand the gastric environment and any dilution effects that normally occur with oral dosing.

FIG. 14 uses the same dosing as FIG. 13: Oral dosing of zanamivir (40 mg/kg) with and without BAC (120 mg/kg) in fasted mice. Fasted animals are dosed by oral gavage with Zanamivir at 40 mg/kg (open circles) or Zanamivir at 40 mg/kg+120 mg/kg BAC-A6 (closed squares). Increasing the dose 10 fold to 40 mg/kg of zanamivir while maintaining the same ratio of BAC to drug (3 to 1 weight to weight) increases the plasma levels of zanamivir greater than 10-fold over that seen with zanamivir alone. In this experiment, an early time point (0.5 hours) is added to determine how rapid the absorption is. Therefore, direct comparison of Cmax peak concentrations cannot be made. However, from these experiments, it is clear that the BAC absorption effect can be seen over at least a 10-fold range of therapeutic dosage.

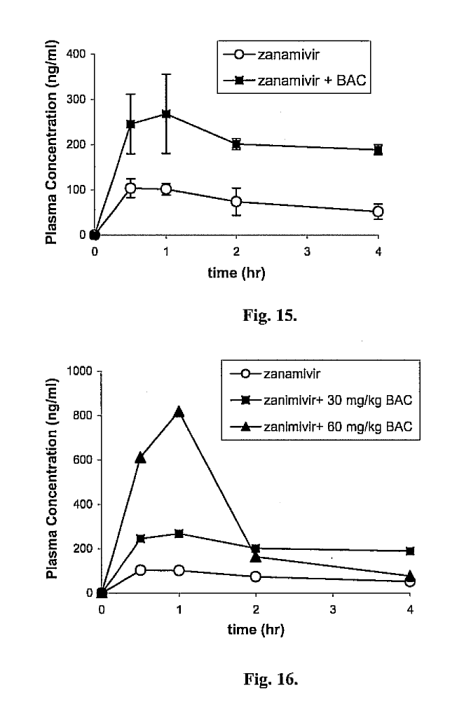

Oral dosing of zanamivir (10 mg/kg) with and without BAC (30 mg/kg) in fed mice is shown in FIG. 15. Fed animals are dosed by oral gavage with zanamivir at 10 mg/kg (open circles) or Zanamivir at 10 mg/kg+30 mg/kg BAC-A6 (closed squares), with plasma zanamivir levels determined at the times indicated. The data represent the average of two mice per data point. Fed mice are dosed by oral gavage with either zanamivir alone or in the presence of BAC at the 3:1 weight to weight ratio. The absorption enhancement by BAC occurs in the fed state, with an approximate 2.5-fold increase in serum concentrations of zanamivir at all time points examined. A diminution of the Cmax and prolongation of the of the absorption and elimination phases is not uncommon in fed state studies. The results clearly show that the BAC effect is evident in the fed state.

Oral dosing of zanamivir (10 mg/kg) at increasing levels of BAC in fed mice is shown in FIG. 16. Fed animals are dosed by oral gavage with zanamivir at 10 mg/kg (open circles), zanamivir at 10 mg/kg+30 mg/kg BAC-A6 (closed squares), and zanamivir at 10 mg/kg+60 mg/kg BAC-A6 (closed triangles), with plasma zanamivir levels determined at the times indicated. Each data point represents the average of two mice. Increasing the BAC to drug ratio to 6:1 weight to weight, dramatically increases the plasma level of zanamivir. Peak serum concentrations of zanamivir are approximated 3-fold higher using a 3:1 w/w ratio and 8-fold higher with the higher 6:1 ratio of BAC-A6:zanamivir.

### Example 21

### The Ability of Various BAC Analogs to Enhance the Oral Bioavailability of the Test Drug Zanamivir in Mice

Oral gavage (PO) dosing solutions of delivery agent compound and zanamivir in water are prepared. The final dosing solutions are prepared by mixing the compound solution with an equal volume of zanamivir stock solution (having a concentration twice the desired final concentration) and diluting to the desired volume (0.2 ml per animal). Representative compound and zanamivir dose amounts are listed in Tables Z5 and Z6.

Male Swiss CFW mice 25-28 g are fasted overnight (unless indicated) and lightly anesthetized with isoflurane by inhalation prior to oral dosing. The mice are administered 0.2 ml of the dosing solution. At each time point, 4 mice are sacrificed from each group and blood is collected by cardiac puncture and plasma fractions prepared. Plasma zanamivir concentrations are quantified by LC/MS/MS analysis.

Tables 5-6 demonstrate the BAC enhancement of zanamivir absorption in mice when the BAC:zanamivir complex is orally administered by gavage. The effect is evident over at least a 10-fold range in dosage level and is seen in both fed and fasted state. Further, altering the ratio of BAC to drug can significantly alter the extent of absorption.

TABLE 5
Oral Gavage (Fasted Mice) Dose 1 mg/kg zanamivir, bile acid
amidyl BACs at 1x (equal molar).

| | | Increase in |
|---|---|---|
| | | concentration |
| | Mean Plasma Zanamivir | above H2O control |
| | Concentration (ng/ml) | (fold) |
| H2O | 55 | 1 |
| control | | |
| A3 | 145 | 3 |
| C8 | 223 | 4 |
| B7 | 228 | 4 |
| A4 | 458 | 8 |
| D11 | 575 | 10 |
| A6 | 891 | 16 |
| E9 | 1227 | 22 |

Plasma samples we taken at 30 min.

TABLE 6
Oral Gavage (Fasted Mice) Dose 4 mg/kg Zanamivir, BACs at 1x (equal molar).

| | | Plasma Zanamivir Concentration | | | |
|---|---|---|---|---|---|
| BAC | | 30 min mean | 60 min mean | AUC 0-1 hr | Increase above |
| Motif | # arginines | (ng/ml) | (ng/ml) | (ng/ml/hr) | H2O control (fold) |
| H2O Control | 0 | 129.36 | 93.54 | 88.06 | 1.00 |
| A6 | 6 | 1058.43 | 138.37 | 563.81 | 6.40 |
| R6 | 6 | 241.29 | 373.63 | 214.05 | 2.43 |
| A9 | 9 | 418.08 | 181.59 | 254.44 | 2.89 |
| C5 | 4 | 211.96 | 124.82 | 137.19 | 1.56 |
| A5 | 5 | 184.08 | 411.02 | 194.79 | 2.21 |
| D11 | 6 | 255.18 | 226.12 | 199.12 | 2.26 |
| B11 | 9 | 857.09 | 460.96 | 543.78 | 6.17 |
| E9 | 3 | 169.84 | 379.28 | 179.74 | 2.04 |
| E13 | 4 | 264.50 | 411.28 | 235.07 | 2.67 |
| A7 | 7 | 160.86 | 86.46 | 102.04 | 1.16 |
| A8 | 8 | 219.95 | 150.82 | 147.68 | 1.68 |
| C8 | 6 | 414.63 | 189.98 | 254.81 | 2.89 |
| C11 | 8 | 330.98 | 281.19 | 235.79 | 2.68 |
| A4 | 4 | 275.09 | 296.16 | 211.59 | 2.40 |
| D9 | 5 | 950.51 | 177.55 | 519.64 | 5.90 |

Plasma samples are taken at 30 and 60 min.

### Example 22

### Effects of BAC Analogs on Alendronate Oral Delivery

Oral gavage (PO) dosing solutions of selected BAC and 14C labeled alendronate are prepared in water. A stock solution of alendronate is made in water. The final dosing solutions are prepared by mixing the compound solution with an equal volume of alendronate stock solution having a concentration twice the desired final concentration) and diluting to the desired volume (0.2 ml per animal).

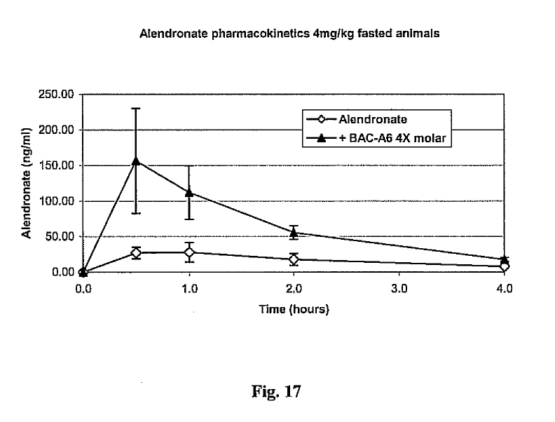

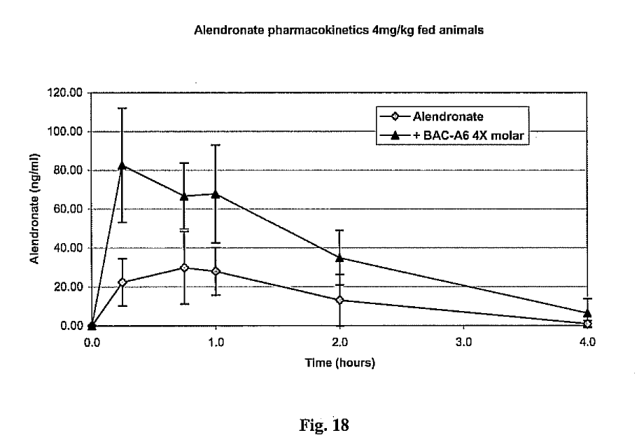

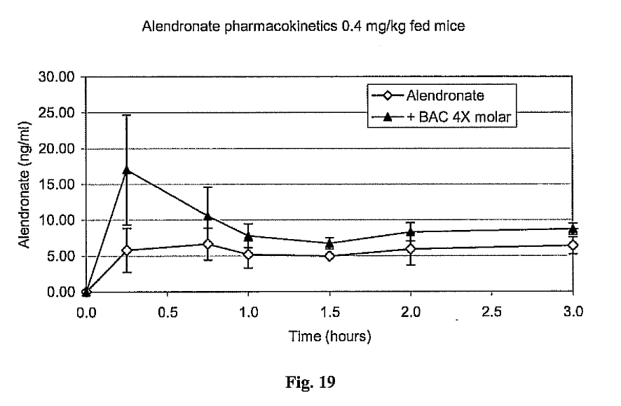

Male Swiss CFW mice 25-28 g are fasted overnight (unless indicated) and lightly anesthetized with isoflurane by inhalation prior to oral dosing. The mice are administered 0.2 ml of the dosing solution. At each time point, 3-4 mice are sacrificed from each group and blood is collected by cardiac puncture and plasma fractions prepared. Plasma alendronate concentrations are quantified by scintillation counting. Representative results comparing fasted and fed animals are depicted in Tables 7-9 and FIGS. 17-19.

TABLE 7
BAC increases absorption of alendronate in fasted mice.

| Time | Alendronate 0x BAC | | | +BAC-A6 4X molar | | |
|---|---|---|---|---|---|---|
| (hours) | ng/ml | +/−S.D.- | AUC | ng/ml | +/−S.D. | AUC |
| 0 | 0.00 | 0.00 | 0.00 | 0.00 | 0.00 | 0.00 |
| 0.5 | 27.19 | 7.98 | 6.80 | 156.66 | 73.73 | 57.60 |
| 1 | 28.11 | 13.92 | 13.83 | 112.16 | 37.75 | 67.20 |
| 2 | 17.97 | 8.30 | 23.04 | 55.85 | 9.58 | 84.00 |
| 4 | 7.83 | 1.60 | 25.81 | 17.71 | 2.77 | 73.56 |
| | | | 69.48 | | | 282.37 |

0x 17.37 ng/ml/hr 4.06 fold
4x 70.59 ng/ml/hr increase
Alendronate to fasted animals
4 mg/kg 100 μg/dose
BAC-A6 4x molar ratio

TABLE 8
BAC increases absorption of alendronate in fed mice.

| | | | | AUC + BAC-A6 4X | | |
|---|---|---|---|---|---|---|
| Time | Alendronate | | | molar | | |
| (hours) | ng/ml | +/−S.D. | AUC | ng/ml | +/−S.D. | AUC |
| 0 | 0.00 | 0.00 | 0.00 | 0.00 | 0.00 | 0.00 |
| 0.25 | 22.39 | 12.20 | 2.80 | 82.61 | 29.49 | 10.33 |
| 0.75 | 29.86 | 18.64 | 13.06 | 66.73 | 17.03 | 37.34 |
| 1 | 27.99 | 12.20 | 7.23 | 67.79 | 25.24 | 16.81 |
| 2 | 13.06 | 13.23 | 20.53 | 34.95 | 14.00 | 51.37 |
| 4 | 0.93 | 1.62 | 14.00 | 6.35 | 7.41 | 41.31 |
| | | | 57.62 | | | 157.15 |

0x 14.40 ng/ml/hr 2.73 fold
4x 39.29 ng/ml/hr increase
Alendronate to fed animals
4 mg/kg 100 μg/dose
BAC-A6 4x molar ratio

TABLE 9
BAC increases absorption of alendronate in fed mice at a lower dosage.

| Time | Alendronate | | | +BAC 4X molar | | |
|---|---|---|---|---|---|---|
| (hours) | ng/ml | +/− | AUC | ng/m | +/− | AUC |
| 0 | 0.00 | 0.00 | 0.00 | 0.00 | 0.00 | 0.00 |
| 0.25 | 5.80 | 3.07 | 0.72 | 17.03 | 7.69 | 2.13 |
| 0.75 | 6.64 | 2.22 | 3.11 | 10.57 | 4.03 | 6.90 |
| 1 | 5.19 | 1.88 | 1.48 | 7.80 | 1.67 | 2.30 |
| 1.5 | 4.95 | 0.17 | 2.54 | 6.71 | 0.78 | 3.63 |
| 2 | 5.92 | 2.22 | 2.72 | 8.31 | 1.28 | 3.76 |
| 3 | 6.40 | 1.20 | 6.16 | 8.73 | 0.78 | 8.52 |
| 4 | 4.71 | 0.51 | 5.56 | 8.47 | 0.91 | 8.60 |
| | | | 22.28 | | | 35.83 |

| | |
|---|---|
| Alendronate | 5.57 ng/ml/hr 1.61 fold increase |
| +4x molar | 8.96 ng/ml/hr |
| bile acid | |
| BAC-A6 | |

Alendronate to fed animals
0.4 mg/kg 10 μg/dose
BAC-A6 4x molar ratio

### Example 23

### Increasing the BAC Molar Ratio Enhances the Oral Bioavailability of Alendronate in Fasted Mice

Oral gavage (PO) dosing solutions of delivery agent compound and 14C labeled alendronate in water are prepared. An additional stock solution of alendronate is made in water. The final dosing solutions are prepared by mixing the compound solution with an equal volume of alendronate stock solution (having a concentration twice the desired final concentration) and diluting to the desired volume (0.2 ml per animal).

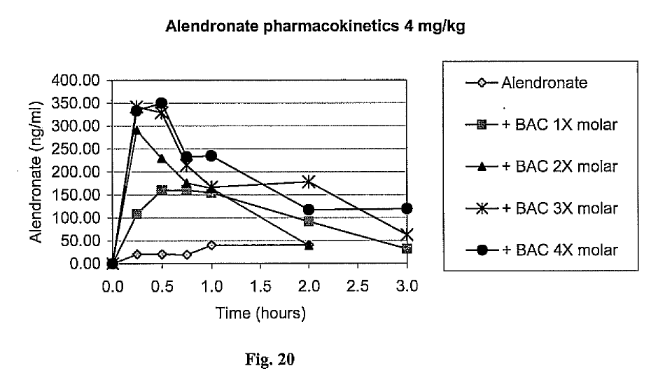

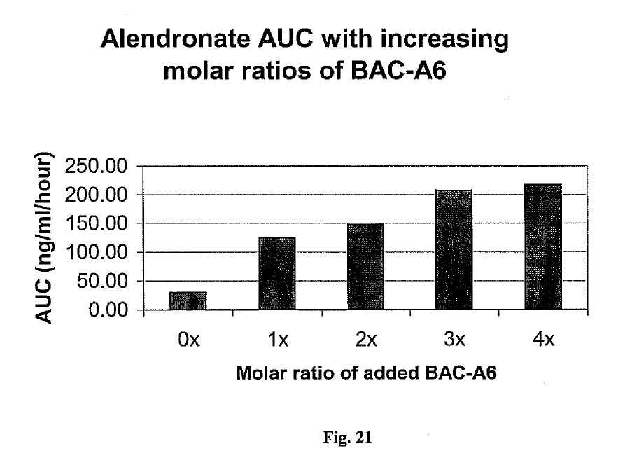

Male Swiss CFW mice 25-28 g are fasted overnight (unless indicated) and lightly anesthetized with isoflurane by inhalation prior to oral dosing. The mice are administered 0.2 ml of the dosing solution. At each time point, 3-4 mice are sacrificed from each group and blood is collected by cardiac puncture and plasma fractions prepared. Plasma alendronate concentrations are quantified by scintillation counting. Representative results are depicted in Table 10 and FIGS. 20-21.

TABLE 10
BAC increases absorption of alendronate in fasted mice in dose response manner.

| Time | Alendronate | | | +BAC 1X molar | | | +BAC 2X molar | | |
|---|---|---|---|---|---|---|---|---|---|
| (hours) | | +/−S.D. | AUC | | +/− | | | +/− | AUC |
| 0 | 0.00 | 0.00 | | 0.00 | 0.00 | 0.00 | 0.00 | 0.00 | 0.00 |
| 0.25 | 20.09 | 1.67 | 2.51 | 108.72 | 5.56 | 13.59 | 290.71 | 56.82 | 36.34 |
| 0.5 | 20.09 | 5.01 | 5.02 | 159.80 | 40.75 | 33.56 | 229.26 | 46.79 | 65.00 |
| 0.75 | 18.91 | 6.68 | 4.87 | 159.80 | 11.11 | 39.95 | 176.08 | 45.12 | 50.67 |
| 1 | 40.18 | 10.03 | 7.39 | 154.56 | 11.11 | 39.29 | 164.26 | 78.55 | 42.54 |
| 2 | 40.18 | 16.71 | 40.18 | 91.69 | 18.52 | 123.12 | 39.00 | 8.36 | 101.63 |
| 3 | | | 59.97 | 31.44 | 3.70 | 249.52 | | | 296.17 |

| +BAC 3X molar | | | +BAC 4X molar | | |
|---|---|---|---|---|---|
| | +/− | | | +/− | |
| 0.00 | 0.00 | 0.00 | 0.00 | 0.00 | 0 |
| 342.04 | 127.29 | 42.75 | 333.02 | 310.40 | 41.6269319 |
| 328.95 | 108.78 | 83.87 | 350.04 | 45.49 | 85.3825137 |
| 214.39 | 48.60 | 67.92 | 232.73 | 66.90 | 72.8471308 |
| 166.93 | 64.80 | 47.66 | 234.62 | 10.70 | 58.4196146 |
| 178.38 | 127.29 | 172.66 | 117.31 | 37.46 | 175.96839 |
| 62.19 | 27.77 | 414.87 | 119.20 | 24.08 | 434.244585 |

| | |
|---|---|
| 0x | 29.99 ng/ml/hr |
| 1x | 124.76 ng/ml/hr |
| 2x | 148.09 ng/ml/hr |
| 3x | 207.43 ng/ml/hr |
| 4x | 217.12 ng/ml/hr |

Alendronate to fasted animals
4 mg/kg 100 μg/dose and (bile acid)
BAC-A6 0x-4x molar ratio

### Example 24

### Effects of BAC Analogs on Methotrexate Bioavailability Following Oral Delivery

Groups of mice are dosed by oral gavage with a solution (0.2 ml) containing methotrexate (MTX) alone, with CDCA, or with the BACs A6 or E9, at increasing molar ratio levels. Plasma is sampled 30 min after dosing. A stock solution of MTX is made in water with NaOH added to solubilize the MTX. Final dosing solutions are prepared by mixing selected BAC solution with an equal volume of MTX stock solution (having a concentration twice the desired final concentration) and diluting to the desired volume (0.2 ml per animal).

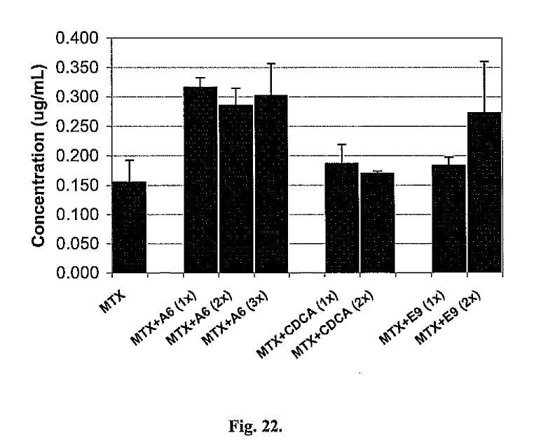

Male Swiss CFW mice 25-28 g are fasted overnight (unless indicated) and lightly anesthetized with isoflurane by inhalation prior to oral dosing. Mice are administered 0.2 ml of the dosing solution. At each time point, 3-4 mice are sacrificed from each group and blood is collected by cardiac puncture and plasma Fractions prepared. Plasma MTX concentrations are quantified by HPLC analysis. Increasing molar ratios of BACs to methotrexate increases serum drug concentrations in mice, as shown in FIG. 22 with plasma concentrations measured 30 min after gavage with 10 mg/kg MTX and 1x, 2× or 3× molar ratios of the BAC_A6 (N-A6 motif of chenoxycholamide) and BAC E9 BAC_A6 (N-E9 motif of chenoxycholamide) or with CDCA. N=3 animals per time point. Error bars indicate standard deviation. The BAC A6 at a 1×, 2× or 3× molar ratios with MTX, increases plasma serum concentration of MTX approximately 2-fold.

### Example 25

### Bone Deposition of Alendronate after Oral Delivery

Oral gavage (PO) dosing stock solutions of delivery agent compound (bile acid) BAC-A6 and 14C labeled alendronate in water are prepared. The final dosing solutions are prepared by mixing the compound solution with an equal volume of alendronate stock solution having a concentration twice the desired final concentration and diluting to the desired volume (0.2 ml per animal, 4 mg/kg alendronate with or without 4× molar ratio of BAC-A6).

Male Swiss CFW mice 25-28 g are fed or fasted overnight and subsequently lightly anesthetized with isoflurane by inhalation prior to oral dosing. Mice are administered 0.2 ml of the dosing solution. 24 hrs after dosing the mice are sacrificed and the tibia collected. Bone alendronate dpm are quantified after oxidation of the samples, collecting the carbon dioxide generated. The amount of 14CO2 is quantified by scintillation counting.

TABLE 11
Bone Deposition of ¹⁴C-alendronate 24 hours after oral dosing.

| | | | |
|---|---|---|---|
| Test condition | mean dpm/mg | bone +/− S.D. | Fold increase |

| | | | |
|---|---|---|---|
| Fasted Alendronate | 3.92 | 2.39 | 1.0 |
| Fasted Alendronate + | 95.90 | 28.08 | 24.5 |
| BAC-A6 | | | |
| Fed Alendronate | 9.51 | 6.61 | 1.0 |
| Fed Alendronate + | 38.27 | 16.52 | 4.0 |
| BAC-A6 | | | |

BAC-A6 increases the absorption and deposition in bone of alendronate after dosing in either fed and fasted animals.

### Example 26

### Effects of BAC_A6 on Cidofovir Bioavailability Following Oral Delivery

Groups of mice are dosed by oral gavage with a solution (0.2 ml) containing cidofovir (4 mg/kg) with and without BAC_A6 (12 mg/kg) in fasted mice. Plasma is sampled 30 min after dosing. Final dosing solutions are prepared by mixing BAC solution with an equal volume of cidofovir stock solution (having a concentration twice the desired final concentration) and diluting to the desired volume (0.2 ml per animal).

Male Swiss CFW mice 25-28 g are fasted overnight, and lightly anesthetized with isoflurane by inhalation prior to oral dosing. Mice are administered 0.2 ml of the dosing solution. At each time point, 3-4 mice are sacrificed from each group and blood is collected by cardiac puncture and plasma fractions prepared, Plasma cidofovir concentrations are quantified by LC/MS/MS analysis. The BAC_A6 at a 1× molar ratio with cidofovir, increases plasma serum concentration of cidofovir approximately 2-3 fold.

Patent applications and publications mentioned in the specification are indicative of the levels of those skilled in the art to which the invention pertains. These applications and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

What is claimed is:

1. A compound having the formula:
RC(O)—X-Z (I)
where RC(O)— is a reaction product of bile acid (5β-CHOLANIC ACID-3α, 7α, -DIOL) or a derivative of the form RCOOH and the derivative is lithocholic acid, deoxycholic acid, chenodeoxycholic acid, ursodeoxycholic acid, ursocholic acid, hyocholic acid, hyodeoxycholic acid, murocholic acid, dehydrocholic acid, 7-ketodeoxycholic acid, dehydrocholic acid, diketocholanic acid, triketocholanic acid, isolithocholic acid, ketolithocholic acid, dehydrolithocholic acid, allocholanic acid, or a salt thereof;
where Z is a 1 to 50 amino acid residue chain having a net charge at physiological pH through at least 20 residue percent basic residues of arginine, lysine, or a combination thereof; at least 20 residue percent acid residues of aspartic acid, glutamic acid, or a combination thereof; or a privileged lysine containing internalization moiety of any one of SEQ ID Nos. 11-18;
where X is a nullity or has a structure prior to reaction with RCOOH of M₁-B-M₂, where M₁ is an amine, CH₂═CH—, iodo-, bromo-, chloro-, or N₂—; where M₂ is amine, CH₂═CH—, iodo-, bromo-, chloro-, or N₂—, carboxylate, thionyl chloride, or acid chloride; and where B is a carbon backbone of 1 to 5 amino acid residues, C₂-C₁₆ alkyl, C₂-C₁₆ alkenyl, C₂-C₁₆ aryl, and C₂-C₁₆ heteroaromatic compounds, where the heteroatom is O, N, or S; and
wherein the compound (I) has a net anionic or cationic charge.

2. The compound of claim 1, where the RCOOH is lithocholic acid, deoxycholic acid, chenodeoxycholic acid, ursodeoxycholic acid, or ursocholic acid or a combination thereof.

3. The composition of claim 1 wherein Z is polycationic.

4. The composition of claim 4 wherein Z contains at least six residues.

5. The compound of claim 1 where Z contains a 3 to 12 amino acid residue chain.

6. The compound of claim 1 where Z is less than 25 total residues in length and more than 35 residue percent arginine or 45 residue percent lysine.

7. The compound of claim 1 where Z is all arginine residues.

8. The compound of claim 1 where Z is a transportan.

9. The compound of claim 1 where Z is a penetratin.

10. The compound of claim 1 where RC(O)— forms and amide bond with X or Z.

11. The compound of claim 10 where X has a substituent pendent from B, wherein the substituent is selected from the group consisting of: a radioactive atom, a magnetic spectroscopically active marker and an organic dye.

12. The compound of claim 1 where X is a the 1 to 5 amino acid residue chain and Z has at least 9 amino acids in the amino acid residue chain.

13. A pharmaceutical composition for increasing bioavailability of a therapeutic in a subject comprising:
the therapeutic having an ionic charge at a physiological pH; and
the compound of claim 1 having an ionic charge opposite that of the ionic charge of the therapeutic at the physiological pH, the compound associated with the therapeutic and present in an amount to neutralize at least in part the ionic charge.

14. The composition of claim 13 wherein the therapeutic is anionic.

15. The composition of claim 13 wherein the therapeutic is selected from the group consisting of 1-dopa, benazeprilat, captoprilat, enalaprilat, fosinoprilat, lisinoprilat, perindoprilat, ouinaprilat, ramiprilat, spiraprilat, trandolaprilat, moexiprilat, cefaclor, cefadroxil, cefamandole, cefatrizine, cefazedone, cefazuflur, cefazolin, cefbuperazone, cefclidine, cefepime, cefetecol, cefixime, cefluprenam, cefmenoxime, cefmetazole, cefodizime, cefonicid, cefoperazone, ceforanide, cefotaxime, cefotefan, cefotiam, cefoxitin, cefpimizole, cefpirome, cefoselis, cefozopran, cefpirome, cefquinome, cefpodoxime, cefroxadine, cefsulodin, cefpiramide, ceflazidime, ceftezole, ceftizoxime, ceftriaxone, cefuroxime, cephacetrile, cephalexin, cephaloglycin, cephaloridine, cephalosporin, cephanone; cephradine, latamoxef, amoxycillin, ampicillin, apalcillin, azidocillin, azlocillin, benzylpencillin, carbenicillin, carfecillin, carindacillin, cloxacillin, cyclacillin, dicloxacillin, epicillin, flucloxacillin, hetacillin, methicillin, mezlocillin, nafcillin, oxacillin, phenethicillin, piperacillin, sulbenicllin, temocillin, ticarcillin, imipenem, meropenem, ertapenem, faropenem, doripenem, danipenem/betamipron, tazobactam, argatroban, melagatran, napsagatran, zanamivir, peramivir, oseltamivir, acametacin, alclofenac, alminoprofen, aspirin (acetylsalicylic acid), 4-biphenylacetic acid, bucloxic acid, carprofen, cinchofen, cinmetacin, clometacin, clonixin, diclenofac, diflunisal, etodolac, fenbufen, fenclofenac, fenclosic acid, fenoprofen, ferobufen, flufenamic acid, flufenisal, flurbiprofin, fluprofen, flutiazin, ibufenac, ibuprofen, indomethacin, indoprofen, ketoprofen, ketorolac, lonazolac, loxoprofen, meclofenamic acid, mefenamic acid, 2-(8-methyl-10,11-dihydro-11-oxodibenz[b,f]oxepin-2-yl)propionic acid, naproxen, nifluminic acid, O-(carbamoylphenoxy)acetic acid, oxoprozin, pirprofen, prodolic acid, salicylic acid, salicylsalicylic acid, sulindac, suprofen, tiaprofenic acid, tolfenamic acid, tolmetin, zopemirac, ciprostene, 16-deoxy-16-hydroxy-16-vinyl prostaglandin E₂, 6,16-dimethylprostaglandin E₂, epoprostostenol, meteneprost, nileprost, prostacyclin, prostaglandins E₁, E₂, or F_{2α}, thromboxane A₂, acrosoxacin, cinoxacin, ciprofloxacin, enoxacin, fleroxacin, flumequine, gatifloxacin, gemifloxacin, grepafloxacin, levofioxacin, lomefloxacin, moxifloxacin, naladixic acid, norfloxacin, ofloxacin, oxolinic acid, pefloxacin, pipemidic acid, piromidic acid, prulifloxacin, rufloxacin, rosoxacin, sitafloxacin, sparfloxacin, temafloxacin, and trovafloxacin, aztreonam, imipenem, meropenem, clorazepate, gabapentin, valproic acid, nateglinide, repaglinide, mitiglinide, furosemide, atorvastatin, cerivastatin, fluvastatin, lovastatin acid, mevastatin acid, pitavastatin, pravastatin acid, rosuvastatin, simvastatin, hydralazine, pemetrexed, nicardipine, pamidronic acid, alendronic acid, ibandronic acid, risedronic acid, zoledronic acid etidronic acid, clodronic acid, tiludronic acid, mycophenolic acid, etoposide phosphate, melphalan, methotrexate, pemetrexed, candesartan, telmisartan, valsartan, aminocaproic acid, acetohydroxamic acid, verteporfin, liothyronine, cromolyn, penicillamine, dimercaptosuccinic acid, ethacrynic acid, montelukast, adefovir, cidofovir, cyclic cidofovir, foscarnet, and tenofovir.

16. The composition of claim 13 wherein the therapeutic is cationic.

17. The composition of claim 16 wherein the therapeutic is selected from the group consisting of: magnesium hydroxide, aluminum hydroxide, calcium carbonate ferrous sulfate, ferrous gluconate, ferrous fumarate, iron-polysaccharide complex, mineral containing multivitamins, sucralfate, amiloride, triamterene, digoxin, morphine, procainamide, quinidine, quinine, ranitidine, trimethoprim, vancomycin, gentamycin, tetracaine, lidocaine, chlorpromazine, propranolol, amantadine, pseudoephedrine: atracurium, mivacurium, cisatracurium, dicyclomine, alfuzosin, terazosin, amlodipine, verapamil, gallopamil, bosentan, cinacalcet, metoprolol, propranolol, timolol, atenolol, pralidoxime, daunorubicin, doxorubicin, epirubicin, idarubicin, mitoxantrone, irinotecan; leuprolide, apraclonidine, clonidine, doxazosin, guanethidine, guanfacine, lofexidine, mecamylamine, methyldopa, moxonidine, prazosin, almotriptan, rizatriptan, darifenacin, methylphenidate, tegaserod, zolmitriptan, ziprasidone; mesylate, doxazosin, phenoxybenzamine, sildenafil, vardenafil, sunitinib, nelfinavir, sertraline, metformin, granisetron, mefloquine; zolpidem, anagrelide, midodrine, selegiline, pergolide, vancomycin, telavancin, oritavancin, venlafaxine, tigecycline, tamoxifen, 4-hydroxytamoxifen, hydralazine, dihydralazine, and pioglitazone.

18. The composition of claim 13 further comprising a pharmaceutically acceptable oral carrier.

19. The composition of claim 13 wherein the amount of present compound of claim 1 present is sufficient to form a micelle around the therapeutic at the physiological pH.

20. A process for increasing bioavailability to a subject of a therapeutic having an ionic charge at physiological pH comprising: administering to the subject in a therapeutically effective amount the therapeutic in concert with the compound of claim 1 having a charge opposite the ionic charge of the therapeutic.

21. The process of claim 20, wherein said administering step is by a route selected from the group consisting of: oral, topical, nasal or inhalational administration.

22. The method of claim 20, wherein the therapeutic is delivered to a subject bloodstream for a period up to twenty-four hours.

23. The method of claim 20, wherein said administering is by parenteral administration.

24. The process of claim 20, wherein further comprising: forming a micelle of the compound of claim 1 around the therapeutic prior to said administering.
